(19) United States
(12) Patent Application Publication
(10) Pub. No.: US 2020/0325543 A1
(43) Pub. Date: Oct. 15, 2020

(54) DIAGNOSTIC METHOD

(71) Applicant: TOLREMO THERAPEUTICS AG, Zurich (CH)

(72) Inventors: Stefanie FLÜCKIGER-MANGUAL, Basel (CH); Wilhelm KREK, Zurich (CH)

(21) Appl. No.: 16/765,419

(22) PCT Filed: Nov. 20, 2018
(86) PCT No.: PCT/EP2018/081897

§ 371 Date: May 19, 2020

(30) Foreign Application Priority Data
Nov. 20, 2017 (EP) 17202584.3

(51) Int. Cl. C12Q 1/6886

(52) U.S. Cl. CPC C12Q 1/6886; C12Q 2600/106; C12Q 2600/118; G01N 2800/44; G01N 2800/52; G01N 2800/60; C12Q 2600/158

(57) ABSTRACT

The present invention is in the field of cancer diagnosis. In particular, the present invention relates to a method for determining in a cancer patient the risk of develop a resistance to chemical substances used in cancer therapy. The invention furthermore provides a novel combination therapy for patients that have been diagnosed to develop drug resistance against chemical substances used for treating cancer.

The present invention is in the field of cancer diagnosis. In particular, the present invention relates to a method for determining in a cancer patient the risk of develop a resistance to chemical substances used in cancer therapy. The invention furthermore provides a novel combination therapy for patients that have been diagnosed to develop drug resistance against chemical substances used for treating cancer.

Genetic alterations of cancer cells often affect genes that are important for cell cycle control, proliferation, differentiation and/or signal transduction. (Hanahan, D., Weinberg, R. A., Cell 100, 57-70 (2000); Hanahan, D., Weinberg, R. A., Cell 144, 646-674 (2011).) Oncogenic activation of MAPK pathway is a signature feature of many human cancers, including melanoma, non-small cell lung cancer (NSCLC) and pancreatic cancer. (Dhillon, A. S. et al., Oncogene 26, 3279-3290 (2007).) For example, 50-70% of melanomas are caused by the BRAF-V600E oncoprotein which activates constitutive MAPK signalling. (Davies, H. et al., Nature 417, 949-954 (2002).) BRAF inhibitors alone or in combination with MEK inhibitors significantly increase patient survival but clinical response is usually of limited duration. (Flaherty, K. T. et al., N. Engl. J. Med. 363, 809-819 (2010); Chapman, P. B. et al., N. Engl. J. Med. 364, 2507-2516 (2011); Hauschild, A. et al., Lancet 380, 358-65 (2012); Flaherty, K. T. et al., N. Engl. J. Med. 367, 1694-1703 (2012); Larkin, J. et al., N. Engl. J. Med. 371, 1867-1876 (2014); Long, G. V. et al., N. Engl. J. Med. 371, 1877-1888 (2014); Robert, C. et al., N. Engl. J. Med. 372, 30-39 (2015); Long, G. V. et al., Lancet 386, 444-451 (2015))

Phenotypic and signalling plasticity as well as the acquisition of novel genetic alterations have been found to be a driving factor in the development of resistance to targeted inhibitors in cancer treatment. (Engelman, J. A. et al., Science 316, 1039-1043 (2007); Kugel, C. H.. et al., Cancer Res. 74, 4122-4132 (2014); Goetz, E. M. et al., Cancer Res. 74, 7079-7089 (2014); Hartsough, E., Shao, Y., Aplin, A. E., J. Invest. Dermatol. 134, 319-325 (2014); Roesch, A. et al., Eur. J. Cancer 59, 109-112 (2016).) Unpredictable inter- and intratumor heterogeneity of the genetic landscape of drug resistant tumors complicates the design of clinical trials to prevent resistance to targeted therapies. (Romano, E., Clin. Cancer Res. 19, 5749-5757 (2013); Shi, H. et al, Cancer Discov. 4, 80-93 (2014); Van Allen, E. M et al., Cancer Discov. 4, 94-109 (2014); Roesch, A., Oncogene 34, 2951-2957 (2015); Shaffer S. M. et al., Nature 564, 431-435 (2017).)

Current approaches to increase patient survival with novel therapies using combinations of targeted inhibitors with additional drugs are oftentimes either used on unselected patient populations or are guided by basal gene expression patterns in cancer cells compared to normal cells or in drug-sensitive cancer cells to drug-resistant cancer cells. (Evans, W. E., Relling, M. V., Nature 429, 464-468 (2014); Glinsky, G. V., Stem Cell Rev. 3, 79-93 (2007); Johannessen, C. M. et al., Nature 504, 38-42 (2013).) Adaptive transcriptional changes in cancer cells in response to drug treatment and intra- and intertumor heterogeneity limit the clinical efficacy of these approaches. One approach to overcome previously observed disadvantages is described in WO 2009/151503. Therein provided is a method to identify a neoplasia as resistant to treatment with a conventional therapy. The method involves the identification of an increased level of various markers in a sample derived from a patient currently undergoing medical treatment of the neoplasia. Thus, the patient already receives conventional treatment. However, cancer treatments can induce changes in cancer cells and thereby lead to drug resistance. (Smith, M. P., Cancer Cell 29, 270-284 (2016).) This means that the described method is unable to identify patients as potentially being at risk to develop resistance before they are exposed to conventional therapy.

There is therefore a need for providing means and methods for reliably determining in a cancer patient the risk of developing a resistance to a given chemical substance, which methods can be used in personalized cancer therapy to identify patients that are amenable to long-term treatment strategies.

The present invention now satisfies this need in that it provides such means and methods, which are more specifically defined in the claims and the following embodiments of the invention.

In one embodiment, the present invention provides a method for determining whether a cell, particularly a cancer or tumor cell, will develop resistance to a chemical substance, wherein the method comprises the following steps:
a) exposure of one or more sample(s) comprising or consisting of cancer or tumor cells obtained from a subject diagnosed with cancer to a chemical substance, wherein the subject diagnosed with cancer has not previously been administered with the said chemical substance;
b) determining the expression level of a gene associated with the development of cancer drug resistance in the one or more sample(s) used in a);
c) determining the expression level of the same gene as in b) in the one or more sample(s) from the subject diagnosed with cancer that is (are) not exposed to the chemical substance used in a);
wherein an elevated expression level determined in b) compared to the expression level determined in c) is indicative of the development of resistance to the chemical substance by a cancer or tumor cell comprised in said sample.

Accordingly, the inventors have unexpectedly and surprisingly found that cells, particularly cancer or tumor cells, comprised in a sample obtained from a subject diagnosed with cancer can be analyzed in vitro to determine whether cells, particularly cancer or tumor cells, comprised in the sample will develop resistance to a chemical substance, in particular a substance used in the treatment of cancer. Specifically, it has not been known or suggested that expression of genes associated with the development of cancer can be induced by chemical substances in an in vitro method. Previous methods known in the art make use of gene signatures that are altered in samples taken from patients after they have already developed resistance to a chemical substance as compared to gene signatures in samples taken from patients before they develop resistance to a chemical substance or while they are still clinically sensitive to said substance. As has been found out by the inventors, drug exposure can drastically influence any subsequent analysis of a potential development of resistance towards chemical substances used in the treatment. The methods of the invention overcome this disadvantage by using samples derived from subjects that do not receive treatment prior to obtaining the one or more sample(s).

In accordance with the above, the invention furthermore provides a method for determining whether a subject previously diagnosed with cancer will develop resistance to a chemical substance used for treating said cancer, wherein the method comprises the steps of
a) exposure of one or more sample(s) comprising or consisting of cancer or tumor cells obtained from the subject diagnosed with cancer to a chemical substance, wherein the subject diagnosed with cancer has not previously been administered with the chemical substance;
b) determining the expression level of a gene associated with the development of cancer drug resistance in the one or more sample(s) used in a);
c) determining the expression level of the same gene as in b) in the one or more sample(s) from the subject diagnosed with cancer that is (are) not exposed to the chemical substance used in a);
wherein an elevated expression level determined in b) compared to the expression level determined in c) is indicative of the development of resistance to the chemical substance by the patient.

Similar to the above method for determining whether a cell, particularly a cancer or tumor cell, will develop resistance to a chemical substance, the method may be employed to determine whether a subject that has been diagnosed with cancer will develop drug resistance to a chemical substance that is used to treat said cancer.

The method of the invention has the surprising and unexpected advantage that it can be employed prior to administering the chemical substance and, thereby avoiding inducing drug resistances. In order to avoid such an induction, samples may be analyzed in vitro whereby the sample(s) have been obtained prior to exposure to the chemical substance. Thus, should the in vitro analysis give rise to an elevated risk of developing drug resistance, induction of resistance by the treatment with such a chemical substance may be prevented by applying alternative treatments with chemical substances which have not shown an increased risk of developing drug resistance in the in vitro analysis according to the present invention or by pursuing a combination treatment or therapy according to the present invention.

In various embodiments, the invention relates to a method according to any one of the preceding embodiments as described herein, wherein the sample is selected from the group consisting of samples obtained from tumor biopsies and from circulating tumor cells in the blood.

In various further embodiments, the invention relates to a method according to any one of the preceding embodiments as described herein, wherein the gene associated with the development of resistance is a gene selected from the group consisting of SOX2, Nanog, OCT4, FGF4, FBX15, FOXP4, KLF9, CD24, CD271, CD36, ITLN2, TNFSF12, NOX3, CLEC7A, ACYAP1, UNC5C, UNC5D, MUC16, VAV3, FOXD3, VGLL3, ALPP, C3, F2R, ENPP2, ETV4, NTNG1, NTRK2, ROBO1 and ROBO2.

In a specific embodiment of the invention, the gene associated with the development of resistance is SOX2.

SRY (sex determining region Y)-box 2, also known as SOX2, is a transcription factor that is essential for maintaining self-renewal, or pluripotency, of undifferentiated embryonic stem cells. The protein is a member of the Sox family of transcription factors, which have been shown to play key roles in many stages of mammalian development. For example, Sox2 controls the branching morphogenesis of the bronchial tree and differentiation of the epithelium of airways in lung development. Under normal conditions, Sox2 is critical for maintaining self-renewal and appropriate proportion of basal cells in adult tracheal epithelium. However, its overexpression gives rise to extensive epithelial hyperplasia and eventually carcinoma in both developing and adult mouse lungs. Moreover, in squamous cell carcinoma, gene amplifications frequently target the 3q26.3 region. The gene for Sox2 lies within this region, which effectively characterizes Sox2 as an oncogene. Sox2 is a key upregulated factor in lung squamous cell carcinoma, directing many genes involved in tumor progression. Sox2 overexpression cooperates with loss of Lkb1 expression to promote squamous cell lung cancer in mice. Its overexpression also activates cellular migration and anchorage-independent growth. Sox2 expression is also found in high gleason grade prostate cancer, and promotes castration-resistant prostate cancer growth. Sox2 has also been shown to be relevant in the development of Tamoxifen resistance in breast cancer. Despite the knowledge about genes associated with the development of cancer, such as SOX2, it has not been previously known that expression of such genes above the native expression level can be induced in vitro by the addition of chemical substances and that the induced overexpression is indicative of the development of resistances towards the applied chemical substance.

In various further embodiments, the invention relates to a method according to any one of the preceding embodiments as described herein, wherein the cancer is melanoma, non-small cell lung cancer, prostate cancer, bile duct cancer, bladder cancer, pancreatic cancer, thyroid cancer, ovarian cancer, colorectal tumor, hairy cell leukemia, acute myeloid leukemia, multiple myeloma, liver cancer, breast cancer, esophageal cancer, head and neck cancer and glioma and wherein the sample obtained from a patient suffering from any one of the above cancers comprises or consists of the respective cancer or tumor cells.

In a specific embodiment, the cancer is melanoma and/or non-small cell lung cancer.

The term “melanoma” as used herein relates to a type of cancer that develops from melanocytes. Melanomas typically occur in the skin but may rarely occur in the mouth, intestines, or eye, all of which are covered by the present invention. The primary cause of melanoma is ultraviolet light (UV) exposure in those with low levels of skin pigment. The UV light may be from either the sun or from other sources, such as tanning devices. It may also develop from moles. Those with many moles, a history of affected family members, and who have poor immune function are at greater risk. A number of genetic defects such as those causing xeroderma pigmentosum also increase risk to develop melanoma. Diagnosis can be by biopsy of any concerning skin lesion. Prevention of melanoma generally involves the use of sunscreen and avoiding UV light. The most common treatment is removal by surgery. However, subjects, in particular subjects with slightly larger cancers, may be tested for spread. Subjects, in particular those in whom melanoma has spread, may require immunotherapy, biologic therapy, radiation therapy, and/or chemotherapy. Despite the various treatment options available in the art, melanoma is still the most dangerous type of skin cancer. Globally, in 2012, it newly occurred in 232,000 people. In 2015 there were 3.1 million with active disease which resulted in 59,800 deaths. Various chemotherapy agents, including temozolomide, dacarbazine (also termed DTIC), immunotherapy (with interleukin-2 (IL-2) or interferon (IFN)), as well as local perfusion, are available. Yet, the overall success in metastatic melanoma is quite limited. IL-2 (Proleukin) has also been shown to be a valuable target for melanoma therapy. Studies have demonstrated that IL-2 offers the possibility of a complete and long-lasting remission in this disease. Therapies for metastatic melanoma include biologic immunotherapy agents including for example ipilimumab, pembrolizumab, and/or nivolumab; BRAF inhibitors, such as vemurafenib and dabrafenib; and MEK inhibitors, such as trametinib and/or cobimetinib, are also available in the treatment of melanoma. Yet, cancer cells, in particular melanoma cells, can develop resistance to available chemical substances used in the treatment. Therefore, it is desirable to provide therapy options that do not lead to the development of resistances or provide methods that reliably predict whether a subject will develop resistance to a chemical substance used in the treatment of melanoma. As detailed above, the present invention provides a reliable method used to determine whether a subject diagnosed with cancer, in particular melanoma, will develop resistance to a chemical substance used in the treatment of cancer, in particular melanoma, such as the chemical substances recited above. Thus, the present invention in some embodiments relates to methods of treating a melanoma in a subject by using chemical substances that have previously not been shown to lead to the development of resistance in said subject. The methods of the present invention are advantageous because the subject has not previously received such treatment and, therefore, did not previously develop resistance.

In other embodiments of the invention, the subject has been diagnosed with non-small-cell lung carcinoma (NSCLC). NSCLC accounts for about 85% of all lung cancers and is thus a major threat. Currently, NSCLCs are relatively insensitive to chemotherapy, compared to small cell carcinoma and other types of cancer. When possible, they are primarily treated by surgical resection with curative intent, although chemotherapy is increasingly being used both pre-operatively (neoadjuvant chemotherapy) and post-operatively (adjuvant chemotherapy). The most common types of NSCLC are squamous cell carcinoma, large cell carcinoma, and adenocarcinoma, but there are several other types that occur less frequently, and all types can occur in unusual histologic variants and as mixed cell-type combinations. More than one kind of treatment is generally used, depending on the stage of the cancer, the individual's overall health, age, response to chemotherapy, and other factors such as the likely side effects of the treatment. The skilled person is well-aware of available treatments. However, after full staging, the NSCLC patient can typically be classified in one of three different categories: patients with early, non-metastatic disease (Stage I, II and select III tumors), patients with locally advanced disease confined to the thoracic cavity (e.g., large tumors, tumors involving critical chest structures or patients with positive mediastinal lymph nodes) or patients with distant metastasis outside of the thoracic cavity. NSCLCs are usually not very sensitive to chemotherapy and/or radiation. However, there are a number of possible chemotherapy agents which can be selected by the person skilled in the art. Most will involve the platinum-based chemotherapy drug cisplatin. Yet, a wide variety of chemotherapies options exist for use in advanced (metastatic) NSCLC. These agents include both traditional chemotherapies like cisplatin which indiscriminately target all rapidly dividing cells as well as newer targeted agents which are more tailored to specific genetic aberrations found within a patient's tumor, At present there are two genetic markers which are routinely profiled in NSCLC tumors to guide further treatment decision making: mutations within EGFR and Anaplastic Lymphoma Kinase. There are also a number of additional genetic markers which are known to be mutated within NSCLC and may impact treatment in the future, including BRAF (gene), HER2/neu and KRAS.

Importantly, roughly 10-35% of NSCLC patients will have drug sensitizing mutations of the EGFR. A number of different EGFR mutations have been discovered, however certain aberrations will result in hyperactive forms of the protein. Patients with these mutations are more likely to have adenocarcinoma histology and be non-smokers or light smokers. These patients have been shown to be sensitized to certain medications which block the EGFR protein known as tyrosine kinase inhibitors specifically, erlotinib, gefitinib or afatinib. SOX2 has been shown to be transcriptionally induced in cultured NSCLC cell lines when exposed to EGFR inhibitors. Reliable identification of mutations in lung cancer needs careful consideration due to the variable sensitivity of diagnostic techniques. In an alternative, up to 7% of NSCLC patients have EML4-ALK translocations or mutations in the ROS1 gene; these patients may benefit from ALK inhibitors that are known to the person skilled in the art. Crizotinib is a known inhibitor of several kinases, specifically ALK, ROS1 and MET.

NSCLC patients with advanced disease who are not found to have either EGFR or ALK mutations may receive bevacizumab which is a monoclonal antibody medication targeted against the vascular endothelial growth factor (VEGF). This is based on an Eastern Cooperative Oncology Group study which found that adding bevacizumab to carboplatin and paclitaxel chemotherapy for certain patients with recurrent or advanced non-small-cell lung cancer (stage IIIB or IV) may increase both overall survival and progression free survival. Another treatment option is the anti-PD-1 agent nivolumab for advanced or metastatic squamous cell carcinoma or pembrolizumab for the treatment of metastatic non-small cell lung cancer (NSCLC) in patients whose tumors express PD-L1 and who have failed treatment with other chemotherapeutic agents.

Pembrolizumab became the first immunotherapy to be used first line in the treatment of NSCLC if the cancer overexpresses PDL1 and the cancer has no mutations in EGFR or in ALK; if chemotherapy has already been administered, then pembrolizumab can be used as a second line treatment but if the cancer has EGFR or ALK mutations, agents targeting those mutations should be used first. Assessment of PDL1 must be conducted with a validated and approved companion diagnostic. However, in all these treatment options, it is desirable to determine the likelihood to develop resistances prior to treatment. In melanoma, MAPK-targeted therapies induce gene expression changes that are similar to the ones detected in tumors which are innately resisant to anti-PD-1 therapy. (Hugo, W., Cell 165, 35-44 (2016).) The methods of the present invention provide such advantageous determination by using one or more sample(s) derived from the patient prior to treatment.

In accordance with the above, in various further embodiments, the invention relates to a method according to any one of the preceding embodiments as described herein, wherein said chemical substance is an inhibitor of a receptor tyrosine kinase (RTK), the EGFR pathway (EGFRi), a checkpoint kinase, an inhibitor of the MAPK pathway (MAPKi) or an agent used in immunotherapy, wherein, preferably, said MAPKi is an inhibitor of B-Raf (BRAFi), an inhibitor of MEK (MEKi), or an inhibitor of ERK (ERKi). The chemical substance may also be an agent used in immunotherapy of cancer, in particular an immuno-oncology agent.

In various specific embodiments
i) said BRAFi is vemurafenib, dabrafenib, encorafenib, LGX818, PLX4720, TAK-632, MLN2480, SB590885, XL281, BMS-908662, PLX3603, R05185426, GSK2118436 or RAF265,
ii) said MEKi is AZD6244, trametinib, selumetinib, cobimetinib, binimetinib, MEK162, R05126766, GDC-0623, PD 0325901, CI-1040, PD-035901, hypothemycin or TAK-733,
iii) said ERKI is ulixertinib, corynoxeine, SCH772984, XMD8-92, FR 180204, GDC-0994, ERK5-IN-1, DEL-22379, BIX 02189, ERK inhibitor (CAS No. 1049738-54-6), ERK inhibitor III (CAS No, 331656-92-9), GDC-0994, honokiol, LY3214996, CC-90003, deltonin, VRT752271, TIC10, astragaloside IV, XMD8-92, VX-11e, mogrol, or VTX11e, and/or
iv) said EGFRi is cetuximab, panitumumab, zalutumumab, nimotuzumab, matuzumab, gefitinib, erlotinib, lapatinib, neratinib, vandetanib, necitumumab, osimertinib, afatinib, AP26113, EGFR inhibitor (CAS No. 879127-07-8), EGFR/ErbB-2/ErbB-4 Inhibitor (CAS No. 881001-19-0), EGFR/ErbB-2 Inhibitor (CAS No. 179248-61-4), EGFR inhibitor II (BIBX 1382,CAS No. 196612-93-8), EGFR inhibitor III (CAS No. 733009-42-2), EGFR/ErbB-2/ErbB-4 Inhibitor II (CAS No. 944341-54-2) or PKC3II/EGFR Inhibitor (CAS No. 145915-60-2).

In particular embodiments of the invention, the chemical substance is an immunotherapy agent, more particular immuno-oncology agent, such as, e.g. an agent targeting CD52, PD-L1, CTLA4, CD20, or PD-1 Agents that may be used in combination with a compound of the present invention include, for example, alemtuzumab, atezolizumab, ipilimumab, nivolumab, ofatumumab, pembrolizumab, rituximab. Further chemical substances are, for example, afatinib, acalabrutinib, alectinib, apatinib, axitinib, bosutinib, cabozantinib, canertinib, crenolanib, cediranib, crizotinib, damnacanthal, dasatinib, entospletinib, entrectinib, erlotinib, foretinib, fostamatinib, gilteritinib, glesatinib, gefitinib, ibrutinib, icotinib, imatinib, linafanib, lapatinib, lestaurtinib, motesanib, mubritinib, nintedanib, nilotinib, ONT-380, pazopanib, quizartinib, regorafenib, rociletinib, radotinib, savolitinib, sitravatinib, semaxanib, sorafenib, sunitinib, savolitinib, sitravatinibg, tesevatinib, vatalanib, vemurafenib or vandetanib.

In another embodiment, the invention relates to the method of any one of the preceding embodiments as described herein, wherein the one or more sample(s) have been obtained by biopsy.

As used herein, a biopsy is a medical test involving extraction of sample cells or tissue(s) usually performed for examination to determine the presence or extent of a disease. The tissue is generally examined under a microscope by a pathologist, and/or is analyzed chemically. When an entire lump or suspicious area is removed, the procedure is called an excisional biopsy. When only a sample of tissue is removed with preservation of the histological architecture of the tissue's cells, the procedure is called an incisional biopsy or core biopsy. When a sample of tissue or fluid is removed with a needle in such a way that cells are removed without preserving the histological architecture of the tissue cells, the procedure is called a needle aspiration biopsy. All different types of biopsy are covered in the present invention unless specified otherwise. Biopsies are most commonly performed for insight into possible cancerous and/or inflammatory conditions, in particular cancer. When cancer is suspected, a variety of biopsy techniques can be applied that are known to the person skilled in the art. An excisional biopsy is an attempt to remove an entire lesion. When the specimen is evaluated, in addition to diagnosis, the amount of uninvolved tissue around the lesion, the surgical margin of the specimen is examined to see if the disease has spread beyond the area biopsied. “Clear margins” or “negative margins” means that no disease was found at the edges of the biopsy specimen. “Positive margins” means that disease was found, and a wider excision may be needed, depending on the diagnosis. When intact removal is not indicated for a variety of reasons, a wedge of tissue may be taken in an incisional biopsy. In some embodiments of the present invention, a sample can be collected by devices that “bite” a sample. A variety of sizes of needle can collect tissue in the lumen (core biopsy). Smaller diameter needles collect cells and cell clusters, fine needle aspiration biopsy. Pathologic examination of a biopsy can determine whether a lesion is benign or malignant, and can help differentiate between different types of cancer.

In contrast to a biopsy that merely samples a lesion, a larger excisional specimen called a resection may come to a pathologist, typically from a surgeon attempting to eradicate a known lesion from a patient. For example, a pathologist would examine a mastectomy specimen, even if a previous nonexcisional breast biopsy had already established the diagnosis of breast cancer. Examination of the full mastectomy specimen would confirm the exact nature of the cancer (subclassification of tumor and histologic “grading”) and reveal the extent of its spread (pathologic “staging”). In another embodiment of the present invention, biopsy is liquid biopsy, i.e. the removal of circulating tumor cells. This method provides a non-invasive alternative to repeat invasive biopsies to evaluate the mutations in cancer and plan individualized treatments. In addition, because cancer is a heterogeneous genetic disease, and excisional biopsies provide only a snapshot in time of some of the rapid, dynamic genetic changes occurring in tumors, liquid biopsies provide some advantages over tissue biopsy-based genomic testing. By detecting and quantifying genomic alterations in CTCs, liquid biopsy can provide real-time information on the stage of tumor progression, treatment effectiveness, and cancer metastasis risk. Within one embodiment of the present invention, it is thus envisaged to use liquid biopsy. Accordingly, in one embodiment of the present invention, biopsy is used to obtain the sample to be analyzed from the subject diagnosed with cancer.

The term “determination” or “determining” is used herein to refer to the evaluation of the risk of a patient for developing resistances to a particular chemical substances, in particular a therapeutic agent. In one embodiment, determination or determining relates to the extent of those resistances. In one embodiment, the determination or determining relates to whether the risk of a patient for developing resistance following treatment, for example treatment with a particular chemical substance/therapeutic agent, is increased/decreased.

The invention also relates to a chemical substance for use in treating cancer in patients determined to develop resistance to said chemical substance using the methods of the invention as described herein in the various embodiments, in combination with an additional chemical substance, wherein said second chemical substance inhibits expression of a gene associated with the development of cancer drug resistance to the first chemical substance or substances.

In one embodiment, the invention relates to the use of one or more chemical substances for treating cancer in patients determined to developed resistance to said chemical substances using the methods of the invention as described herein in the various embodiments in combination with an additional chemical substance which inhibits the expression of one or more genes associated with the development of cancer drug resistance to the first chemical substance or substances.

Further comprised by the present invention is a product containing a combination of one or more chemical substances for treating cancer in patients determined to induce resistance to said chemical substances using the methods of the invention as described herein in the various embodiments and an additional chemical substance which inhibits the expression of one or more genes associates with the development of cancer drug resistance to the first chemical substance.

Said cancer to be treated, in one embodiment, may be non-melanoma skin cancer, esophagogastric adenocarcinoma, glioblastoma, bladder cancer, bladder urothelial carcinoma, esophagogastric cancer, melanoma, non-small cell lung cancer, endometrial cancer, cervical adenocarcinoma, esophageal squamous cell carcinoma, breast cancer, head and neck squamous cell carcinoma, germ cell tumor, small cell lung cancer, ovarian cancer, soft tissue sarcoma, hepatocellular carcinoma, colorectal adenocarcinoma, cervical squamous cell carcinoma, cholangiocarcinoma, prostate cancer, upper tract urothelial carcinoma, diffuse glioma, colorectal cancer, ampullary carcinoma, adrenocortical carcinoma, head and neck cancer, renal clear cell carcinoma, hepatobiliary cancer, glioma, non-Hodgkin lymphoma, mesothelioma, salivary gland cancer, renal non-clear cell carcinoma, miscellaneous neuroepithelial tumor, pheochromocytoma, thymic tumor, multiple myeloma, renal cell carcinoma, bone cancer, pancreatic cancer, leukemia, peripheral nervous system tumors, thyroid cancer, B-lymphoblast leukemia, monoclonal B-cell lymphocytosis, lymphoma, hairy cell leukemia, acute myeloid leukemia, Wilms tumor, in particular melanoma and non-small cell lung cancer. The above diseases typically exhibit a mutation incidence of more than 3% of RTKs (EGFR, ERBB2, ERBB3, ERBB4, PDGFA, PDGFB, PDGFRA, PDGFRB, KIT, FGF1, FGFR1, IGF1, IGFR, VEGFA, VEGFB, KDR) and/or MAPK pathway members (KRAS, HRAS, BRAF, RAF1, MAP3K1/2/3/4/5, MAP2K1/2/3/4/5, MAPK1/3/4/6/7/8/9/12/14, DAB, RASSF1, RAB25).

The chemical substance for use of the invention may be an inhibitor of a receptor tyrosine kinase (RTK), the EGFR pathway (EGFRi) or an inhibitor of the MAPK pathway (MAPKi), wherein, preferably, said MAPKi is an inhibitor of B-Raf (BRAFi), an inhibitor of MEK (MEKi), or an inhibitor of ERK (ERKi). In a preferred embodiment of the invention, the chemical substance may be a BRAFi, in particular vemurafenib, dabrafenib, encorafenib, LGX818, PLX4720, TAK-632, MLN2480, 5B590885, XL281 or RAF265, and/or a MEKi, in particular AZD6244, trametinib, selumetinib, cobimetinib, binimetinib, MEK162, R05126766, GDC-0623, PD 0325901, CI-1040 or TAK-733, and/or an ERKi, in particular ulixertinib, SCH772984, XMD8-92, FR 180204, GDC-0994, ERK5-IN-1, DEL-22379, BIX 02189, ERK inhibitor (GAS No. 1049738-54-6), ERK inhibitor III (CAS No. 331656-92-9), GDC-0994 or VTX11e, and/or an EGFRi, in particular cetuximab, panitumumab, zalutumumab, nimotuzumab, matuzumab, gefitinib, erlotinib, lapatinib, AP26113, EGFR inhibitor (CAS No. 879127-07-8), EGFR/ErbB-2/ErbB-4 Inhibitor (CAS No. 881001-19-0), EGFR/ErbB-2 Inhibitor (CAS No. 179248-61-4), EGFR inhibitor II (BIBX 1382,CAS No. 196612-93-8), EGFR inhibitor III (CAS No. 733009-42-2), EGFR/ErbB-2/ErbB-4 Inhibitor II (CAS No. 944341-54-2) or PKCβII/EGFR Inhibitor (CAS No. 145915-60-2).

The second chemical substance, which may be administered simultaneously or sequentially with the first chemical substance inhibits the expression of a gene associated with the development of cancer drug resistance. In a preferred embodiment the second chemical substance inhibits a gene selected from the group consisting of SOX2, Nanog, OCT4, FGF4, FBX15, FOXP4, KLF9, CD24, CD271, CD36, ITLN2, TNFSF12, NOX3, CLEC7A, ACYAP1, UNC5C, UNC5D, MUC16, VAV3, FOXD3, VGLL3, ALPP, C3, F2R, ENPP2, ETV4, NTNG1, NTRK2, ROBO1 and ROBO2. It is preferred that the second chemical substance inhibits the expression of SOX2.

In this respect, it has surprisingly and unexpectedly found out by the inventors that existing approved drugs may be used to inhibit genes associated with drug resistance. Accordingly, in a preferred embodiment of the invention, the chemical substance for use in the treatment of cancer, preferably one of the above inhibitors, may be combined with an existing approved drug in order to prevent that the patient develops drug resistance towards the chemical substance used for treating the cancer. Accordingly, the invention, in one embodiment, relates to the chemical substance for use in treating cancer of the invention, wherein the second chemical substance is selected from the group consisting of cetrimonium bromide, idarubicin.hcl, neratinib (hki-272), benzyl isothiocyanate, vorinostat, emetine dihydrochloride, daunorubicin hydrochloride, dactinomycin, quisinostat (jnj26481585), niclosamide, doxorubicin, pci-24781 (abexinostat), lanatoside c, panobinostat (lbh589), salinomycin, sodium, broxaldine, teniposide, pracinostat (sb939), azacitidine, homoharringtonine, acrisorcin, tolonium chloride, radotinib, amodiaquine dihydrochloride, benzethonium chloride, chidamide, cudc-101, selamectin, tetrandrine, belinostat (pxd101), etravirine (tmc125), amcinonide, oxibendazole, acetyl-1-leucine, chloroxine, napabucasin, resminostat, idoxuridine, tioguanine, cycloheximide, trifluridine, betamethasone 17,21, dipropionate, dovitinib (tki-258) dilactic acid, colchicine, mocetinostat (mgcd0103), sunitinib, pelitinib (ekb-569), pimavanserin, efloxate, tg101348 (sar302503), clobetasol propionate, methylprednisolone sodium succinate, dichlorisone acetate, albendazole, entinostat (ms-275), flunisolide, artenimol, aminacrine, flumethasone, rocilinostat (acy-1215), bronopol, gramicidin (gramicidin a shown), abamectin (ivermectin bla shown), disulfiram, difluprednate, acetriazoic acid, isoflupredone acetate, ly2835219, perhexiline maleate, metergoline, formestane, monensin sodium, floxuridine, prednicarbate, dexamethasone sodium phosphate, leflunomide, halobetasol propionate, sirolimus, ipriflavone, nintedanib (bibf 1120), pyrvinium, pamoate, rufloxacin hydrochloride, fosbretabulin (combretastatin a4 phosphate (ca4p)), disodium, triamcinolone diacetate, otenabant (cp-945598) hcl, aprotinin, fluticasone propionate, amuvatinib (mp-470), methylbenzethonium chloride, fenbendazole, bupivacaine hydrochloride, betamethasone, flumethazone pivalate, thioguanine, tegaserod maleate, prednisolone acetate, chlorindione, hydrocortisone hemisuccinate, dexamethasone acetate, fludrocortisone acetate, ivermectin, proflavine hemisulfate, lansoprazole, cerdulatinib (prt062070, prt2070), salifungin, halcinonide, fudosteine, terfenadine, fluocinonide, hexetidine, artesunate, fluocinolone acetonide, rifampin, triamcinolone, zolpidem, ethopropazine, hydrochloride, regorafenib (bay 73-4506), terazosin hydrochloride, tanshinone iia-sulfonic sodium, nocodazole, triclosan, clopidol, sorafenib tosylate, sulfisomidine, methylene blue, crizotinib (pf-02341066), proscillaridin a, dexibuprofen, triflupromazine hydrochloride, piribedil hydrochloride, carmofur, swertiamarin, sultamicillin tosylate, ginsenoside rc, etofibrate, cetylpyridinium chloride, rabeprazole sodium, alizapride hydrochloride, methyl aminolevulinate.hcl, topiroxostat, disodium clodronate tetrahydrate, amoxapine, bedaquiline(tmc207; r207910), octenidine, ecabet sodium, apigenin, glycopyrrolate iodide, sodium montmorillonite, hydrocortisone. Further chemical substances envisaged as inhibitor of genes associated with drug resistance include substances targeting ARRB1, ATXN7L3, CBX1, CREBBP, CTBP2, CUL3, DDB2, FMR1, FOXO1, KDM4B, KMT2E/MLL5, NIPBL, OGFOD1, RBX1, SF3B1, SFPQ, SRSF1, SSRP1, YWHAZ or ZMYND11, such as, for example, barbadin, CCS1477, SGC-CBP30, CPI-637, PF-CBP1, ICG,001,PRI-724, A-485, C646, 4-methylthio-2-oxobutyric acid (MTOB), HIPP derivatives, cyclic peptide CP61, NSC95397, 2-(hydroxyimino)-3-phenylpropanoic acid and 4-chloro and 3-chloro analogues thereof, MLN4924, AS1842856, JIB-04, EP-5676, N-oxalylglycine (NOG), pyridine-2,4-dicarboxylate (2,4-PDCA), pladineolide B, IDC16, CBL0137, difopein, or R18.

The invention also covers all further features shown in the figures individually, although they may not have been described in the previous or following description. Also, single alternatives of the embodiments described in the figures and the description and single alternatives of features thereof can be disclaimed from the subject matter of the other aspect of the invention.

Furthermore, in the claims the word “comprising” does not exclude other elements or steps, and the indefinite article “a” or “an” does not exclude a plurality. A single unit may fulfil the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

As used in this specification and the appended claims, the singular forms “a”, “an”, and “the” include plural referents unless the context clearly dictates otherwise. Thus, for example, reference to “a compound” includes one or more compounds.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease; (b) inhibiting the disease, i.e. arresting its development; or (c) relieving the disease, i.e. causing regression of the disease.

A “patient” or “subject” for the purposes of the present invention is used interchangeably and meant to include both humans and other animals, particularly mammals, and other organisms. Thus, the methods are applicable to both human therapy and veterinary applications. In the preferred embodiment the patient or subject is a mammal, and in the most preferred embodiment the patient or subject is a human.

A cell-based screening method may be used to identify compounds effective in the treatment of cancer characterized by cells that are characterized by an over-activated signalling pathway, in a combination medicament with an inhibitor of said signalling pathway comprising the steps of a) providing a cell that carries an activating mutation or amplification in a gene encoding a protein comprised in said signalling pathway; b) bringing said cell in contact with an inhibitor of said signalling pathway; and a test compound; c) determining an expression level of a gene associated with the development of resistance selected from the group consisting of SOX2, Nanog, OCT4, FGF4, FBX15, FOXP4, KLF9, CD24, CD271, CD36, ITLN2, TNFSF12, NOX3, CLEC7A, ACYAP1, UNC5C, UNC5D, MUC16, VAV3, FOXD3, VGLL3, ALPP, C3, F2R, ENPP2, ETV4, NTNG1, NTRK2, ROBO1 and ROBO2. It is preferred that the second chemical substance inhibits the expression of SOX2; and d) assigning to said test compound a score, wherein said score is high if said expression level of SOX2 and/or another gene associated with the development of resistance is below a predetermined threshold, said threshold corresponding to an expression level of SOX2 and/or another gene associated with the development of resistance in a control cell treated solely with said inhibitor of said signalling pathway; said score is low if said expression level of SOX2 and/or another gene associated with the development of resistance is equal to or above said predetermined threshold.

A plurality of cells may be employed simultaneously in step a); said plurality of cells is submitted to step b) together; the average expression level of SOX2 and/or another gene associated with the development of resistance is determined for said plurality of cells; and a high score is assigned to said test compound if said is average expression level of SOX2 and/or another gene associated with the development of resistance is below the average expression level of SOX2 and/or another gene associated with the development of resistance in control cells treated solely with said inhibitor of said signalling pathway.

In particular, a plurality of cells may be employed simultaneously in step a); said plurality of cells is submitted to step b) together; a single cell is evaluated as “SOX2 positive” if said SOX2 expression level and/or another gene associated with the development of resistance is above an expression level determined for an untreated cell and a ratio of “SOX2 positive” cells to total cells is determined for said plurality of cells; and d) a high score is assigned to said test compound if said ratio determined for cells treated with said test compound is below said ratio determined for control cells treated solely with inhibitor of said signalling pathway.

The skilled person is well-aware of methods suitable to determine the expression level of SOX2 and/or another gene associated with the development of resistance . In one specific embodiment, the expression level is determined by analysing protein expression and/or mRNA expression. However, any method using information derived from the genome, transcriptome and/or proteome may be employed in the present invention. For example, the expression level can be determined on protein level (of SOX2 and/or another gene associated with the development of resistance) and can be directly visualized and quantified by the use of labels, in particular antibody-mediated staining. The expression level can also be determined on mRNA level (of SOX2 and/or another gene associated with the development of resistance) by direct visualization using in situ hybridization. Using such techniques, individual molecules can be quantified.

In certain embodiments, a gene associated with the development of resistance is selected from the group comprising Nanog, OCT4, FGF4, FBX15, FOXP4, KLF9, CD24, CD271, CD36, ITLN2, TNFSFI2, NOX3, CLEC7A, ACYAPI, UNC5C, UNC5D, MUC16, VAV3, FOXD3, VGLL3, ALPP, C3, F2R, ENPP2, ETV4, NTNG1, NTRK2, ROBO1 and ROBO2.

Accordingly, within the present invention, gene expression levels may be determined using any technique known in the art, for example methods based on hybridisation of polynucleotides (mRNA transcripts), methods based on sequencing polynucleotides or amplifying polynucleotides.

Quantification of mRNA gene transcript in a sample may be performed using, without limitation, northern blotting, in situ hybridisation, RNAse protease assays, PCR based methods such as reverse transcription polymerase chain reaction (RT-PCR) and real time quantitative POT qRT-PCR. Alternatively, antibodies with binding specificity to nucleic acid duplexes may be used to determine mRNA levels. Microarray techniques using specific binding members for RNAs of interest, for example cDNA or oligonucleotide probes specific for RNAs of interest or antibodies specific for mRNA of interest wherein the specific binding members are plated or arrayed on a substrate, for example a glass slide or a microchip substrate can be used. The specific binding members may be provided on the substrate at an addressable location and the number of addressable locations can vary from, for example at least three, at least 10, at least 50, at least 100, at least 1000 or at least 10,000 or more. In embodiments the number of addressable locations can vary from less than 1000, less than 100, less than 50, less than 10, or less than 5. In such embodiments the sample is contacted with the array and the arrayed specific binding members can form detectable interactions with targets in the sample. The interactions may be detected using suitable labels. Where oligonucleotide probes are utilised, under appropriate conditions the oligonucleotide probes can “hybridise” to a target nucleic acid sequence to form base-paired duplexes with nucleic acid molecules that have a complementary base sequence. Hybridisation conditions resulting in particular degrees of stringency will vary depending on the nature of the hybridisation method and the composition and length of the hybridising nucleic acid sequences.

Stringent hybridisation occurs when a nucleic acid binds a target nucleic acid with minimal background. Typically, to achieve stringent hybridisation, temperatures of around 1° C. to about 20° C., more preferably 5° C. to about 20° C. below the Tm (melting temperature at which half the molecules dissociate from their partner) are used. However, it is further defined by ionic strength and pH of the solution. Suitable hybridisation conditions would be known to those of skill in the art, and exemplary hybridisation conditions are: Very high stringency (detects sequences that share at least 90% identity)—hybridisation 5× SSC at 65° C. for about 16 hours, High stringency (detects sequences that share at least 80% identity)—hybridisation 5×-6× SSC at 65° C. for 16 hours, and Low stringency (detects sequences that share at least 50% identity)—hybridisation 6× SSC at room temperature to 55 ° C. for 20 to 30 minutes.

An example of a highly stringent wash condition is 0.15 M NaCl at 72° C. for about 15 minutes. An example of a stringent wash condition is 0.2× sodium chloride and sodium citrate (SSC) wash at 65° C. for 15 minutes (see, Sambrook and Russell, infra, for a description of SSC buffer for example 20× SSC made by dissolving 175.3 g of NaCl and 88.9 g of sodium citrate in 800 ml distilled water. Adjusting pH to pH7.0 with HCl (IM) and adjusting volume to IL with distilled water). Often, a high stringency wash is preceded by a low stringency wash to remove background probe signal. An example of a medium stringency wash for a duplex of, for example, more than 100 nucleotides, is 1× SSC at 45° C. for 15 minutes. An example of a low stringency wash for a duplex of, for example more than 100 nucleotides, is 4-6× SSC at 40° C. for 15 minutes. For short probes (for example about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (for example, >50 nucleotides).

The methodology used in PCR methods for example RT-PCR and POT and RT-PCR will be well known to those skilled in the art.

Some methods may require the isolation of RNA from a sample. Such isolation techniques are known in the art and may utilise commercially available RNA isolation kits from manufacturers such as Qiagen.

Determination of protein expression Immunohistochemistry (IHC) and ELISA are techniques useful for detecting protein expression. Antibodies or binding fragments of antibodies (monoclonal or polyclonal) may be used in the disclosed methods and kits. Antibodies can be detected by direct labelling of the antibodies or by using a second antibody which is specific for the primary antibody which has binding specificity for the target. The second antibody can be labelled with a detectable moiety or can be conjugated to a hapten (such as a biotin or the like) wherein the hapten is detectable by a detectably labelled cognate hapten binding molecule, for example streptavidin horseradish peroxidise.

The binding specificity of antibodies (antibodies with binding specificity to a particular antigen, e.g. SOX2 and/or another gene associated with the development of resistance) can be established using Western blotting, in parallel with immunohistochemical analysis of formalin-fixed, paraffin-embedded cell lines mimicking the handling of the primary tumours (as described by O'Brien et al., 2007, International Journal of Cancer, 120:1434-1443)

Alternatively, proteins may be detected using aptamers (for example a single stranded nucleic acid molecule (such as, DNA or RNA) that assumes a specific, sequence dependent shape and binds to FKBPL protein with high affinity and specificity), mirror image aptamers (SPIEGELMER™), engineered nonimmunuoglobulin binding proteins, for example nonimmunoglobulin binding proteins based on scaffolds including fibronectin (ADNECTINS™), CTLA-1 (EVIBODIES™), lipocalins (ANTICALINS™), protein A domain (AFFIBODIES™) or the like. In embodiments, an aptamer may comprise less than 100 nucleotides, less than 75 nucleotides, less than 50 nucleotides, for example 25 to 50 nucleotides, 10 to 50 nucleotides, 10 to 100 nucleotides.

In particular embodiments, an array may be provided comprising protein sequences, including SOX2 protein or fragments of SOX2 protein or antibodies with binding specificity to SOX2 protein or fragments thereof. These protein sequences or antibodies can be conjoined to a substrate. Changes in protein expression can be detected by, for example, measuring the level of SOX2 protein and/or another gene associated with the development of resistance in a sample which binds to antibodies with binding specificity to SOX2 protein and/or another gene associated with the development of resistance when the sample to be tested is brought into contact with the array.

In certain embodiments, a gene associated with the development of resistance is selected from the group comprising Nanog, OCT4, FGF4, FBX15, FOXP4, KLF9, CD24, CD271, CD36, ITLN2, TNFSFI2, NOX3, CLEC7A, ACYAP1, UNC5C, UNC5D, MUC16, VAV3, FOXD3, VGLL3, ALPP, C3, F2R, ENPP2, ETV4, NTNG1, NTRK2, ROBO1 and ROBO2.

Suitable substrates for use in an array and array formats would be known to those of skill in the art.

In particular embodiments IHC samples can be analysed using an automated image analysis system, so as to provide a blinded analysis. For this, whole-slide digital images can be first captured at 2Ox using a ScanScope XT Slide Scanner (Aperio Technologies). Secondly, a positive pixel count algorithm (Aperio Technologies) can be used to develop a quantitative scoring model for SOX2 expression. Statistical analysis of tissue microarray-derived data can be carried out using the v2 test for trend, Fisher's exact and Mann-Whitney tests for comparison of SOX2 expression and Kaplan-Meier plots can be used for survival analysis and the curves compared using the log-rank test. Cox proportional hazards regression can be used to estimate proportional hazard ratios and conduct multivariate analyses as described previously. All calculations can be performed with SPSS v11.0 (SPSS, IL). In addition, to facilitate generation of discrete multi-marker test, fluorescently-tagged antibodies (carrying non-overlapping fluorophores) then additional relevant biomarkers can be used simultaneously. Advantageously a recently developed fluorescent scanning system from Aperio, for example, the ScanScope FL system could be used. This assay method would provide a further layer of sophistication by providing more quantitative analysis than that afforded by conventional brightfield imaging. As will be appreciated, the methods of detecting the expression of SOX2, the location of SOX2 in a cell or the activity of SOX2 may be applicable in relation to any of the methods of the invention described herein or claimed.

Preferred features and embodiments of each aspect of the invention are as for each of the other aspects mutatis mutandis unless context demands otherwise.

A nucleic acid molecule is said to be complementary with another nucleic acid molecule if the two molecules share a significant number of complementary nucleotides to form a stable duplex or triplex when the strands bind (hybridise) to each other, for example by forming Watson-Crick base pairs. Complementarity can be described as a percentage of the proportion of base pairs between two nucleic acid molecules within a specific region of two molecules.

By contact is meant to bring an agent into close proximity with another agent such that both agents can interact with each other. For example an antibody or other binding member may be brought into close proximity with a protein in a sample and where the antibody has binding specificity for the protein the antibody will bind the protein. Alternatively, a first nucleic acid may be brought into close proximity with a second complementary nucleic acid (a primer with a target sequence) and can be incubated such that binding may be detected or amplification of the target sequence may occur.

By detect is meant determining if an interaction between two agents for example two proteins or two nucleic acids is present or absent. This may include quantification. Detection may include the use of an agent which is capable of detection (a label) using for example spectrophotometry, flow cytometry, or microscopy. Exemplary labels include radioactive isotopes (such as ³H, ¹⁴C, ¹⁵N, ³⁵S, ⁹⁰V, ⁹⁹Tc, ¹¹¹Ln, ¹²⁵I₁Or ¹³¹I), fluorophores (such as fluorescein, fluorescein isothiocyanate, rhodamine or the like), chromophores, ligands, chemiluminescent agents, bioluminescent agents (such as luciferase, green fluorescent protein (GFP) or yellow fluorescent protein), enzymes that can produce a detectable reaction product (such as horseradish peroxidise, luciferase, alkaline phosphatase, beta-galactosidase) and combinations thereof.

By specific binding is meant a particular interaction between one binding partner and another binding partner, for example a primer and a target sequence or a protein specific antibody and a protein. Interactions between one binding partner and another binding partner may be mediated by one or more, typically more than one, non-covalent bonds. An exemplary way of characterising specific binding is by a specific binding curve.

In a further embodiment of the method of the invention, the method comprises a step wherein the cell cycle phase is determined, in particular cell cycle arrest is detected, in said cell treated with said inhibitor of said signalling pathway and said test compound, and wherein a high score is assigned to said test compound if said cell undergoes cell cycle arrest.

In a particular embodiment of the method of the invention, the signalling pathway is the MAPK or EGFR pathway. In this embodiment of the invention, the cancer may be characterized by cancer cells that carry an activating mutation or amplification in a gene encoding a protein comprised in the MAPK or EGFR pathway, in particular an activating mutation or amplification in NRAS, KRAS, HRAS, BRAF, MEK, ERK, ROS, ALK, MET, KIT or EGFR. The cancer may then be selected from non-melanoma skin cancer, esophagogastric adenocarcinoma, glioblastoma, bladder cancer, bladder urothelial carcinoma, esophagogastric cancer, melanoma, non-small cell lung cancer, endometrial cancer, cervical adenocarcinoma, esophageal squamous cell carcinoma, breast cancer, head and neck squamous cell carcinoma, germ cell tumor, small cell lung cancer, ovarian cancer, soft tissue sarcoma, hepatocellular carcinoma, colorectal adenocarcinoma, cervical squamous cell carcinoma, cholangiocarcinoma, prostate cancer, upper tract urothelial carcinoma, diffuse glioma, colorectal cancer, ampullary carcinoma, adrenocortical carcinoma, head and neck cancer, renal clear cell carcinoma, hepatobiliary cancer, glioma, non-Hodgkin lymphoma, mesothelioma, salivary gland cancer, renal non-clear cell carcinoma, miscellaneous neuroepithelial tumor, pheochromocytoma, thymic tumor, multiple myeloma, renal cell carcinoma, bone cancer, pancreatic cancer, leukemia, peripheral nervous system tumors, thyroid cancer, B-lymphoblast leukemia, monoclonal B-cell lymphocytosis, lymphoma, hairy cell leukemia, acute myeloid leukemia, Wilms tumor in particular melanoma and non-small cell lung cancer. In a particular embodiment of the method of the invention, the cell provided in step a is selected from a melanoma cell or a non-small cell lung cancer cell carrying a BRAF mutation, in particular the BRAF-V600E or BRAF-V600K mutation, and a non-small cell lung cancer cell carrying a EGFR mutation, amplification or overexpression.

In the methods of the present invention, the inhibitor of the signalling pathway may for example be selected from an inhibitor of the EGFR pathway (EGFRi) and an inhibitor of the MAPK pathway (MAPKi), wherein in particular said MAPKi is selected from an inhibitor of B-Raf (BRAFi), an inhibitor of MEK (MEKi), and an inhibitor of ERK (ERKi). Within the present invention, such inhibitors may be vemurafenib, dabrafenib, encorafenib, LGX818, PLX4720, TAK-632, MLN2480, SB590885, XL281 or RAF265, said MEKi may be AZD6244, trametinib, selumetinib, cobimetinib, binimetinib, MEK162, RO5126766, GDC-0623, PD 0325901, CI-1040 or TAK-733, said ERKi may be ulixertinib, SCH772984, XMD8-92, FR 180204, GDC-0994, ERK5-IN-1, DEL-22379, BIX 02189, ERK inhibitor (CAS No. 1049738-54-6), ERK inhibitor III (CAS No. 331656-92-9), GDC-0994 or VTX11e, or said EGFRi may be cetuximab, panitumumab, zalutumumab, nimotuzumab, matuzumab, gefitinib, erlotinib, lapatinib, AP26113, EGFR inhibitor (CAS No. 879127-07-8), EGFR/ErbB-2/ErbB-4 Inhibitor (CAS No. 881001-19-0), EGFR/ErbB-2 Inhibitor (CAS No. 179248-61-4), EGFR inhibitor II (BIBX 1382,CAS No. 196612-93-8), EGFR inhibitor III (CAS No. 733009-42-2), EGFR/ErbB-2/ErbB-4 Inhibitor II (CAS No. 944341-54-2) or PKCβII/EGFR Inhibitor (CAS No. 145915-60-2).

In the context of the present invention, and in particular of the method of the invention, the term “protein comprised in a signalling pathway” relates to molecules that interact in a cell to control a specific cellular function, such as proliferation, differentiation or apoptosis. Molecules that are comprised in one signalling pathway are part of a concerted activation cascade. Upon a stimulus, the first molecule in the pathway activates one or several downstream molecules. The activation is passed on until the last molecule in the activation chain is activated and the cellular function is carried out. With respect to the MAPK pathway, this relates to specific ligands, receptors and downstream transcription factors selected from the group comprising NGF, NRG, BDNF, NT3/4, EGF, FGF, PDGF, CACN, TrkA/B, EGFR, FGFR, PDGFR, ROS, ALK, MET, KIT, GFB2, SOS, HRAS, KRAS, NRAS, RasGRF, RasGRP, CNasGEF, PKC, PKA, Rap1, G12, Gap1m, NF1, p120GAF, RafB, ARAF, BRAF, CRAF, Mos, MEK1, MEK2, MP1, ERK1, ERK2, PTP, MKP, Tau, STMN1, cPLA2, MNK1/2, RSK2, CREB, Elk-1, Sap1a, c-Myc, SRF5 and c-fos.

The term “activating mutation” in the context of the present invention relates to an alteration in the nucleotide sequence of a gene that results in an increased activity of the gene product. The increased activity can be due to enhanced enzymatic activity, prolonged half-life or overexpression of the gene product.

The term “gene under transcriptional control of SOX2” in the context of the present invention relates to a gene characterized by a first expression level if SOX2 is not expressed in the same cell, and a second expression level if SOX2 is expressed in the same cell. The first expression level is lower than the second expression level. In certain embodiments, a gene associated with the development of resistance is selected from the group comprising Nanog, OCT4, FGF4, FBX15, FOXP4, KLF9, CD24, CD271, CD36, 1TLN2, TNFSF12, NOX3, CLEC7A, ACYAP1, UNC5C, UNC5D, MUC16, VAV3, FOXD3, VGLL3, ALPP, C3, F2R, ENPP2, ETV4, NTNG1, NTRK2, ROBO1 and ROBO2.

In certain embodiments, the invention provides a cell-based method of determining in a cancer patient whether the patient is at risk of developing a resistance to chemical substances or compounds effective in the treatment of cancer. The cancer is characterized by cells that are characterized by an over-activated signalling pathway. The over-activation may be caused by an activating mutation in or amplification of a gene encoding a protein that acts in the signalling pathway. The skilled person is aware that over-activation of a signalling pathway may also be caused by an inhibiting mutation in or a deletion of a gene encoding an inhibitor of the signaling pathway such as NF1. The compounds are effective in the treatment of the cancer in a combination medicament comprising an inhibitor of the respective over activated signalling pathway,

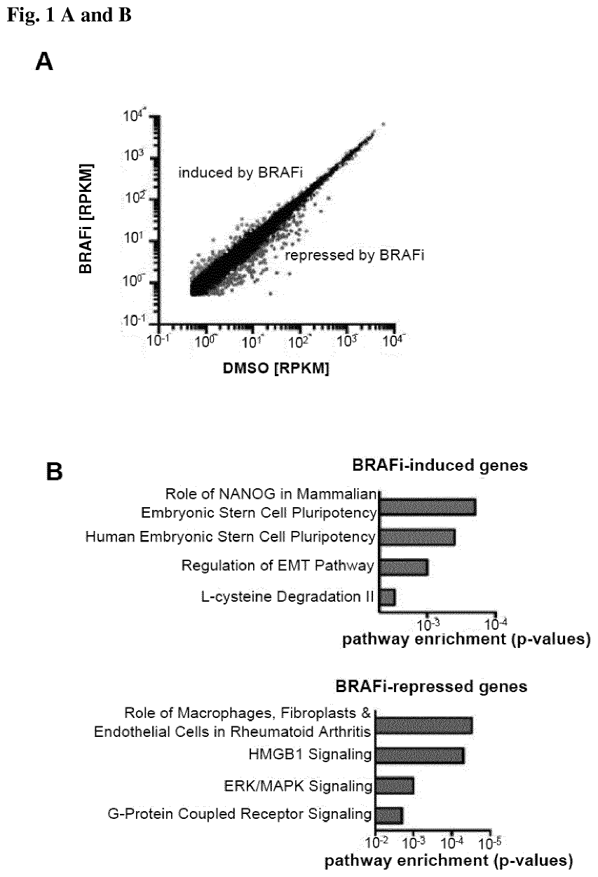

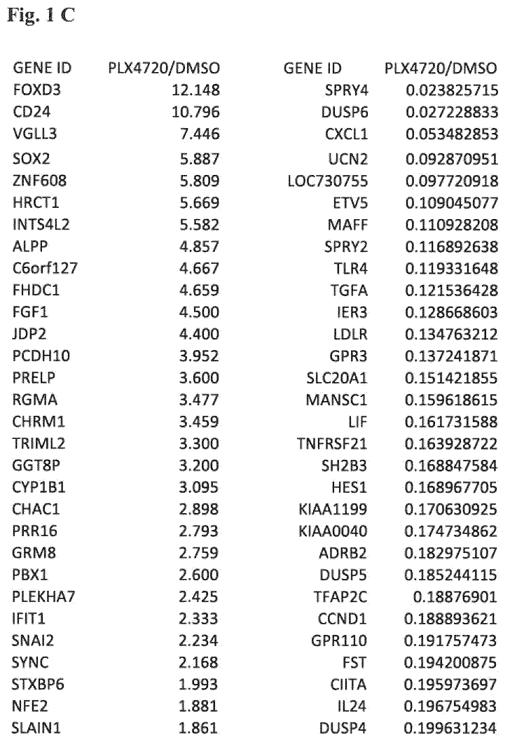

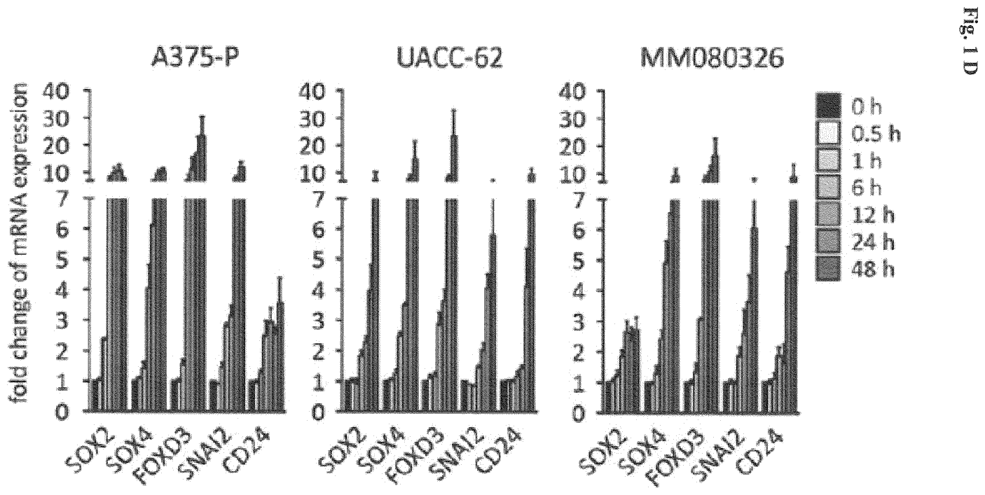

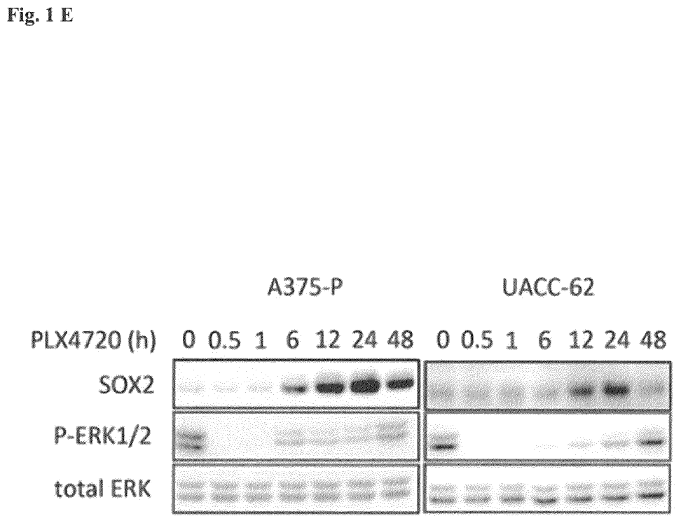

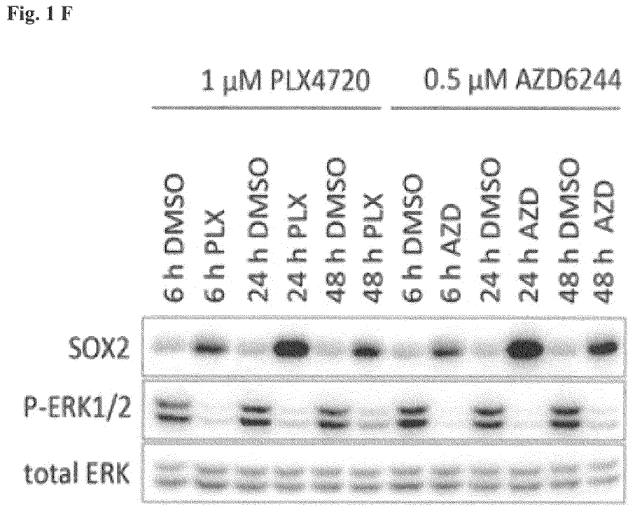

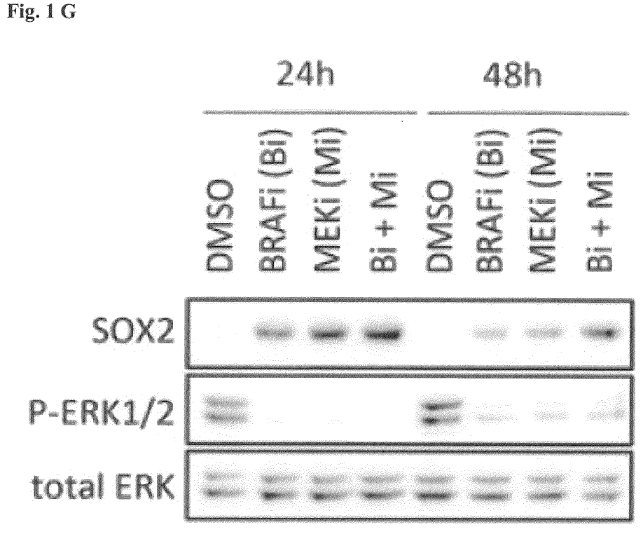

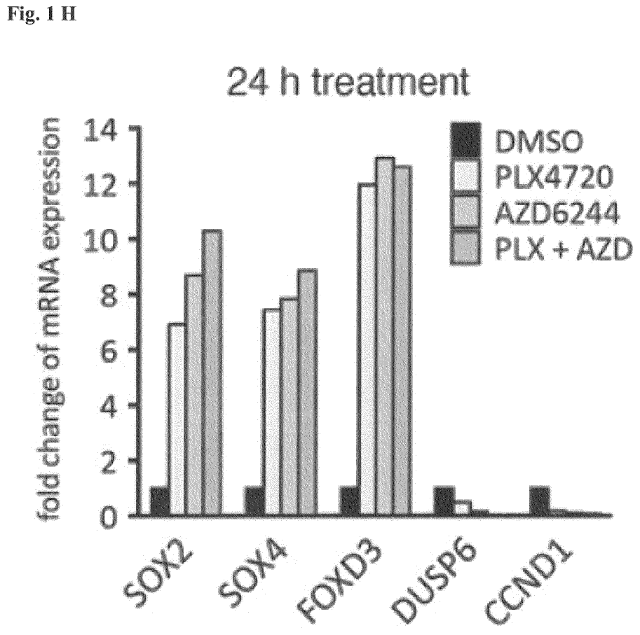

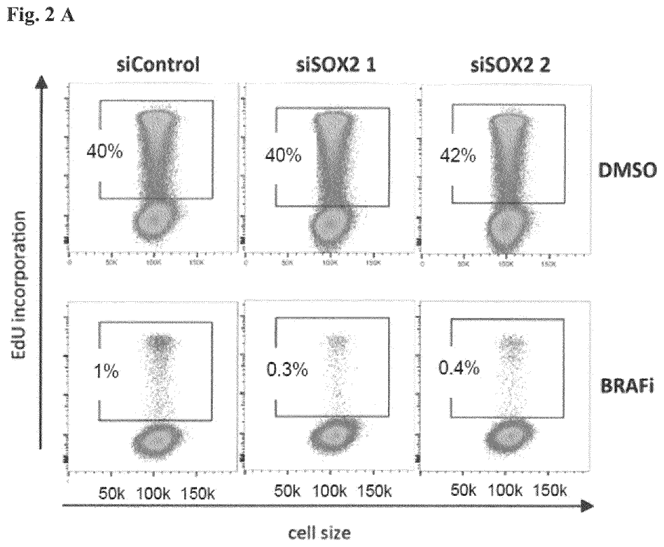

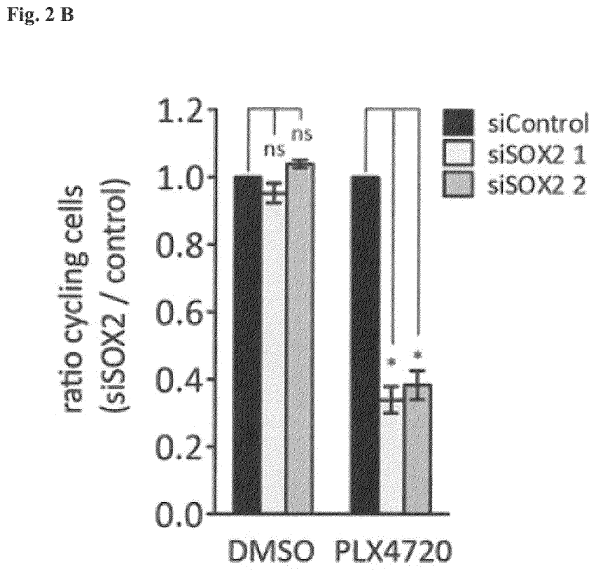

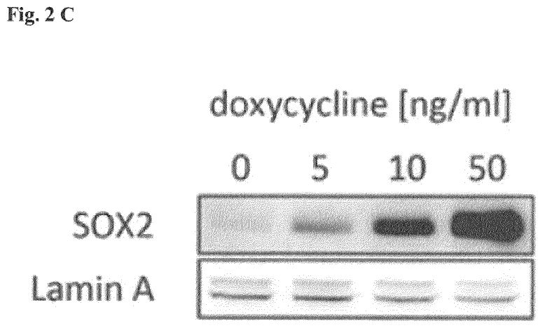

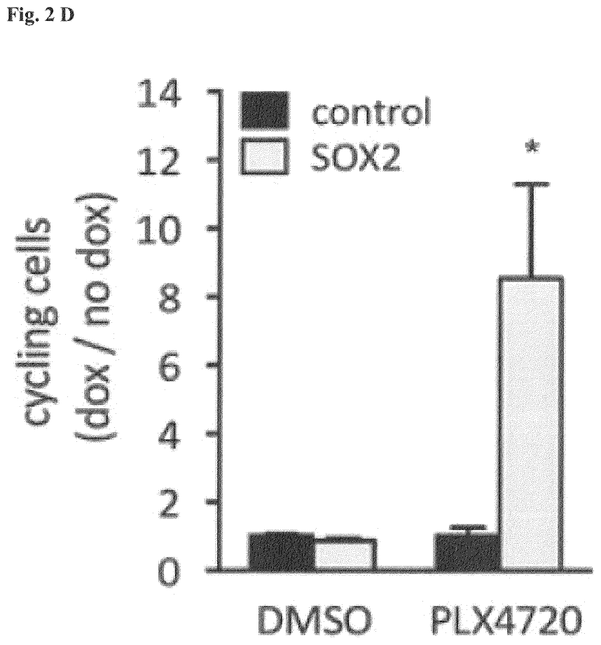

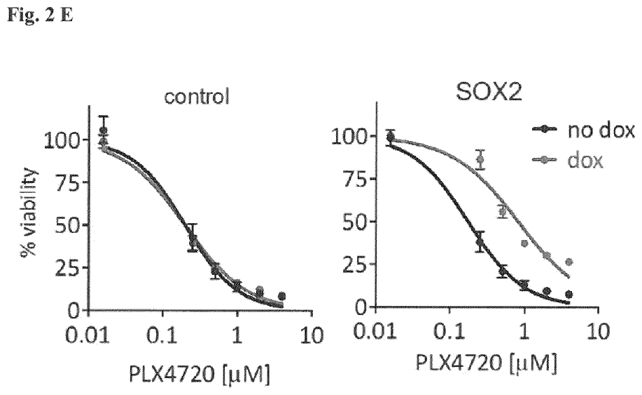

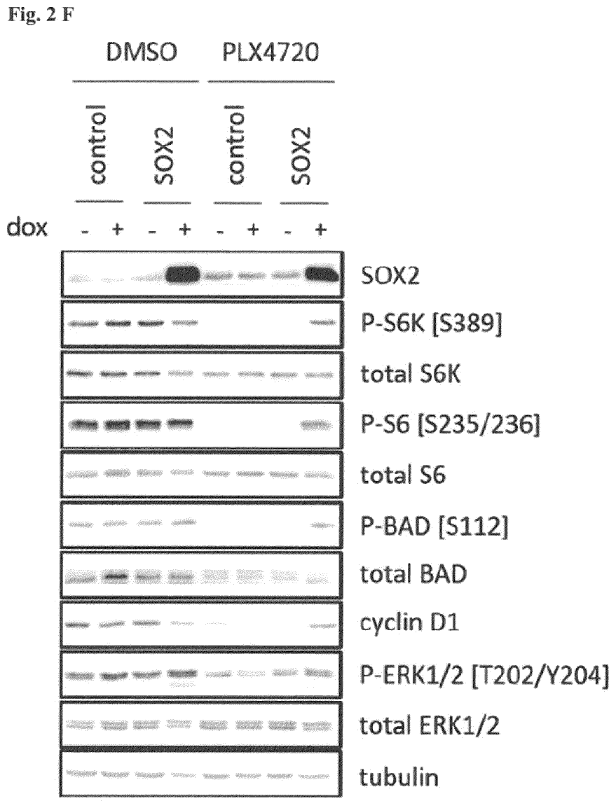

In this respect, the inventors surprisingly found that inhibitors of mutant BRAF and MEK (hereafter collectively referred to as MAPK inhibitors, MAPKi) induce an acute transcriptional response (i.e. adaptive resistance programs; ARPs) in melanoma cells within hours of drug application. These ARPs involve the transcriptional induction of SOX2-dependent sternness, axon guidance and EMT (epithelial-to-mesenchymal transition) genes, leading to the generation of a pool of drug-tolerant cells (FIG. 1). These drug-tolerant cells can seed the development of acquired resistance in the long-term. The number of drug-tolerant cells can be significantly reduced by siRNA mediated knockdown of SOX2, the master regulator of sternness and main driver of MAPKi-induced ARPs (FIG. 2). As a consequence, preventing the induction of ARPs by blunting MAPKi-induced SOX2 in the clinical context will be of highest therapeutic value to prevent acquired drug resistance thus extending the durability of clinical-used MAPK inhibitors.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention.

Unless otherwise indicated, established methods of recombinant gene technology were used as described, for example, in Sambrook, Russell “Molecular Cloning, A Laboratory Manual”, Cold Spring Harbor Laboratory, N.Y. (2001) which is incorporated herein by reference in its entirety.

A number of documents including patent applications, manufacturer's manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

FIG. 1 shows the induction of a sternness and EMT signature upon acute treatment with MAPKi analysed by RNA-Seq. (A) A375-P cells were treated with PLX4720 (BRAFi) or DMSO for 6 h, RNA was isolated and RNA-Seq was performed. Scatter plot shows all genes with minimal normalized abundance of ≥0.5 RPKM. Differentially expressed genes (DEGs) selected for pathway analysis are represented by red and blue dots depending on the direction of the transcriptional change (red genes are induced upon PLX4720, blue genes are repressed upon PLX4720). (B) Bar graphs showing the five top pathways with significant enrichment of the analyzed DEGs. Longer bars indicate stronger significance. (C) Top deregulated genes upon treatment with PLX4720 (left column). Top genes induced and repressed by PLX4720 are shown in separate lists. (D) Indicated cell lines were treated with 1 μM PLX4720 or 0.01% DMSO (0 h) for the indicated times and the expressional changes of selected genes associated with stemness/EMT were confirmed by qPCR. All cell lines expressed similar adaptive gene programs upon BRAF inhibition. Data shown represent mean of independent biological triplicates ±SEM. (E) Western blot analysis of the same experiment as shown in (D). As key player in the sternness signature, SOX2 levels were assessed. P-ERK1/2 levels served as positive treatment controls. Total ERK1/2 levels served as loading control. (F) A375-P cells were treated with 1 μM PLX4720, 0.5 μM AZD6244 (MEK1/2 inhibitor) or 0.01% DMSO for the indicated times and SOX2 levels were assessed by Western blot analysis. Note that inhibition of BRAF and inhibition of MEK1/2 induce similar SOX2 levels, suggesting this to be a general feature of oncogenic MAPK inhibition. (G) A375-P cells were treated with 1 μM PLX4720 (BRAFi), 0.5 μM AZD6244 (MEKi), a combination of both drugs or 0.01% DMSO for the indicated times and SOX2 levels were assessed by Western blot analysis. Note that BRAFi and MEKi had comparable effects on SOX2 levels, with the combination of both drugs leading to slightly higher SOX2 levels. (H) A375-P cells were treated with 1 μM PLX4720 (BRAFi), 0.5 μM AZD6244 (MEKi), a combination of both drugs or 0.01% DMSO for the 24 h and the expressional changes of selected genes associated with stemness/EMT were confirmed by qPCR. Note that BRAFi and MEKi had comparable effects on transcript levels, with the combination of boths drugs being slightly more efficient at transcript induction. For all Western blots, individual time points were assessed in at least two independent experiments. Representative data is shown.

FIG. 2 shows the protective effect of SOX2 from MAPKi-induced anti-proliferative effects. (A) A375-P cells were transfected with 100 nM siRNA targeting SOX2 or a negative control siRNA. Cells were then treated with PLX4720 (BRAFi) or DMSO for 48 h. 2 h before harvesting, cells were pulsed with 3 uM EdU to label cycling cells. EdU-positive cells were detected using flow cytometry. Note that SOX2 knockdown has no effect on cell proliferation when BRAF is active (DMSO) but reduces the pool of drug-tolerant, cycling cells in the BRAFi condition. Representative data is shown. (B) Quantification of experiment (A). For each condition, percentage of cycling cells upon siRNA knockdown of SOX2 was normalized to the negative control siRNA. Data shown represent mean of independent biological triplicates ±SEM. (C) A375-P cells stably transduced with pTRIPZ-SOX2 vector were treated with the indicated concentrations of doxycycline for three days or with 1 μM PLX4720 (PLX) for 24 h. SOX2 overexpression was confirmed by Western blot analysis. Lamin A served as loading control. Individual concentrations were assessed in at least two independent experiments. Representative results are shown. (D) A375-P/pTRIPZ-control or -SOX2 cells were pre-treated with 0 ng/ml (negative control) or with 50 ng/ml doxycycline for 24 h, and subsequently with DMSO or PLX4720 for an additional 48 h. 2 h before harvesting, cells were pulsed with 3 uM EdU to label cycling cells. EdU-positive cells were detected using flow cytometry. Data shown is normalized to the percentage of cycling cells in the no doxycycline condition. Shown is mean of independent biological triplicates ±SEM. (E) A375-P/pTRIPZ-control or -SOX2 cells were pre-treated with 0 ng/ml (negative control) or with 50 ng/ml doxycycline for 24 h, and subsequently with serial dilutions of PLX4720 for an additional four days. Cell viability was assessed by PrestoBlue assay and IC50 values were calculated. Note that doxycycline has no effect on pTRIPZ-control cells but shifts the survival curve to the right in the pTRIPZ-SOX2 cells, indicating decreased drug sensitivity when SOX2 is overexpressed. IC50 values are stated above the survival curves. Data shown is normalized to DMSO-treated cells. Error bars indicate SEM of three independent experiments. (F) A375-P/pTRIPZ-control or -SOX2 cells were pre-treated with 0 ng/ml (negative control) or with 50 ng ml doxycycline for 24 h, and subsequently with DMSO or PLX4720 for an additional 48 h. Survival signaling was assessed by Western blot. Survival signaling via S6K and BAD is repressed by PLX4720 but rescued when SOX2 is overexpressed by doxycycline treatment.

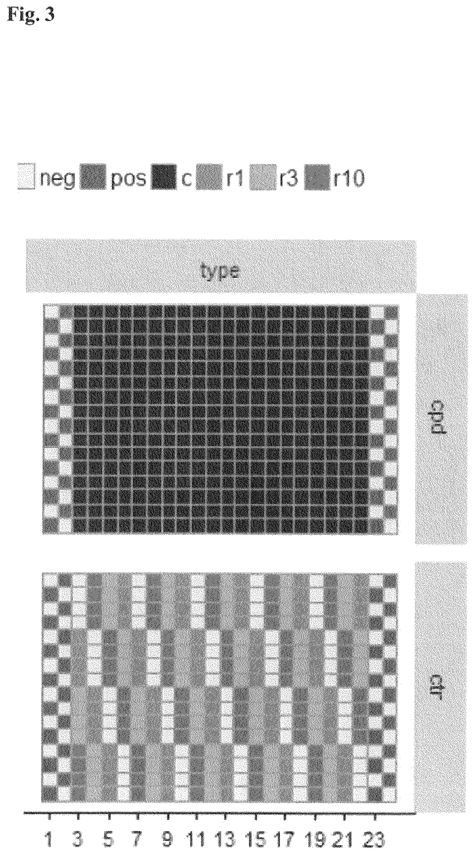

FIG. 3 Heatmap representation of compound well allocation. neg: DMSO; pas: 5 uM Entinostat; c: 5 uM test compound; r1: 1 uM Entinostat; r3: 3 uM Entinostat; r10: 10 uM Entinostat.

FIG. 4 Log-transformed signal of control wells showing the original fluorescence fl2 and fl3 as well as their ratio y. The y-axis scaling is between the 1% and 99% quantile.

FIG. 5 Log-transformed signal of control wells before (y) and after normalization (norm). The y-axis scaling is between the 1% and 99% quantile.

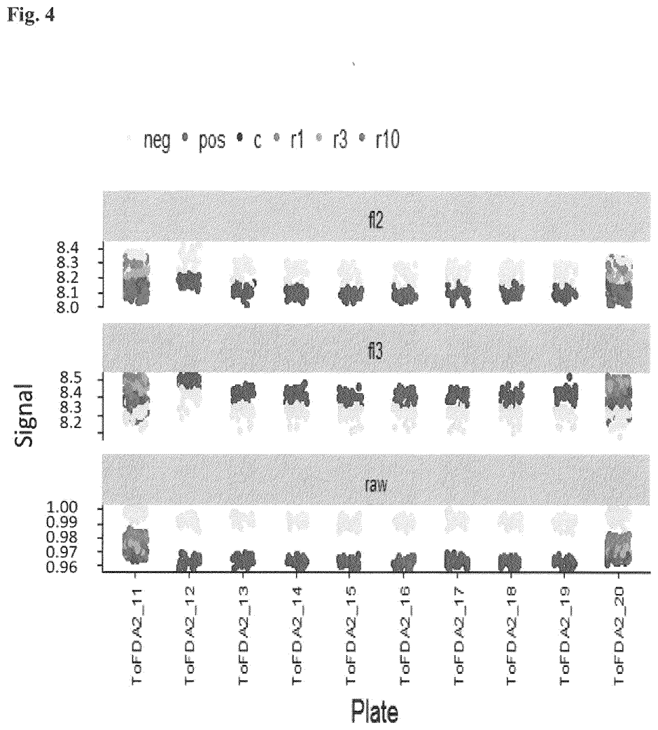

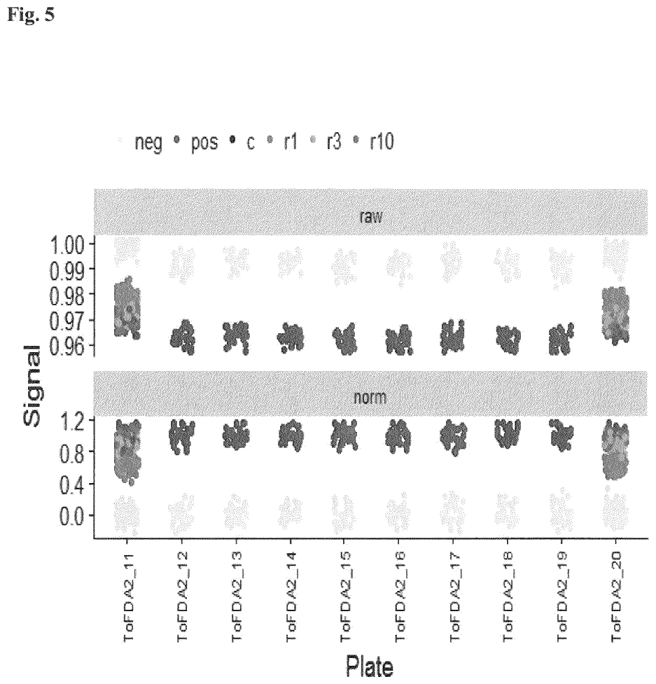

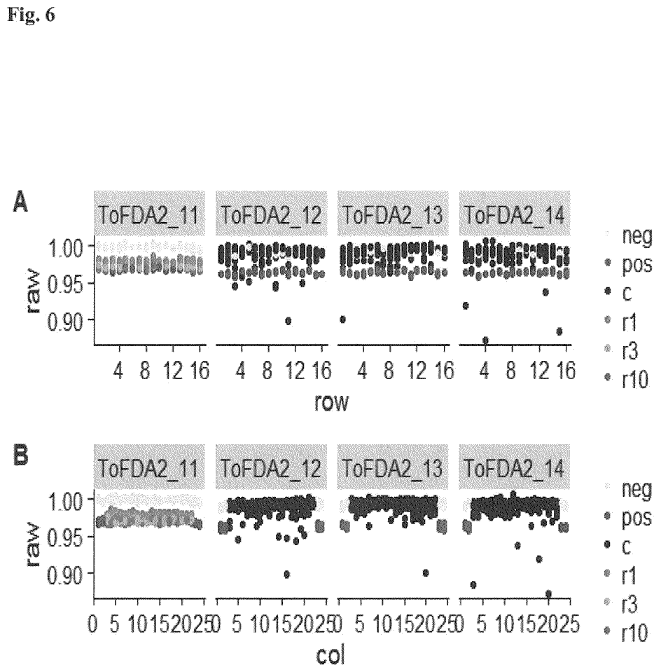

FIG. 6 Raw signal versus column or row number for the first 4 plates. Systematic row or column effects can be identified if present.

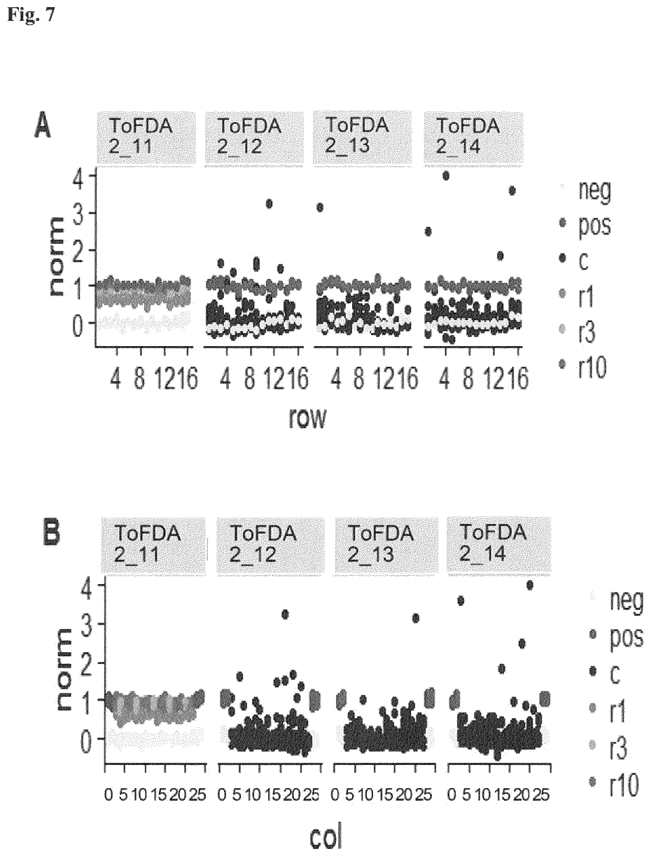

FIG. 7 Normalized (A, B) and geometry-corrected signal (C, D) versus row (A, C) or column number (B, D) for the first 4 plates. Systematic row or column effects should not be visible any more.

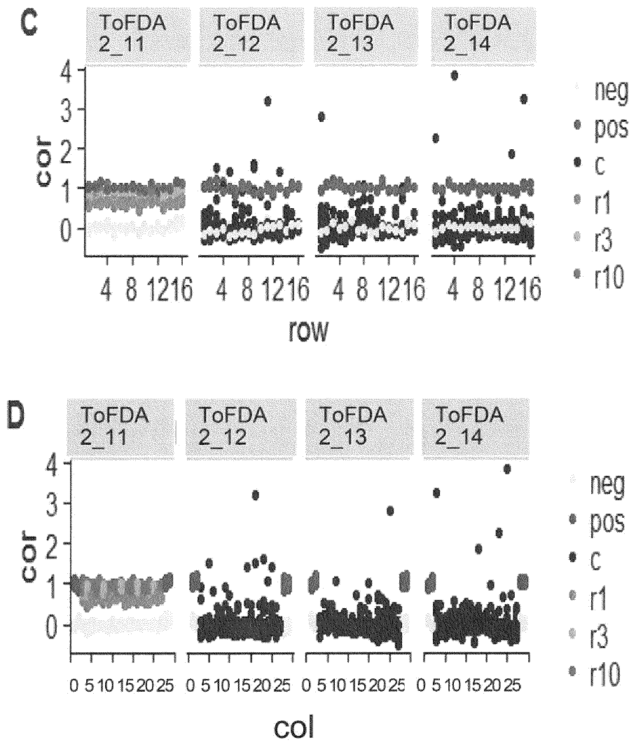

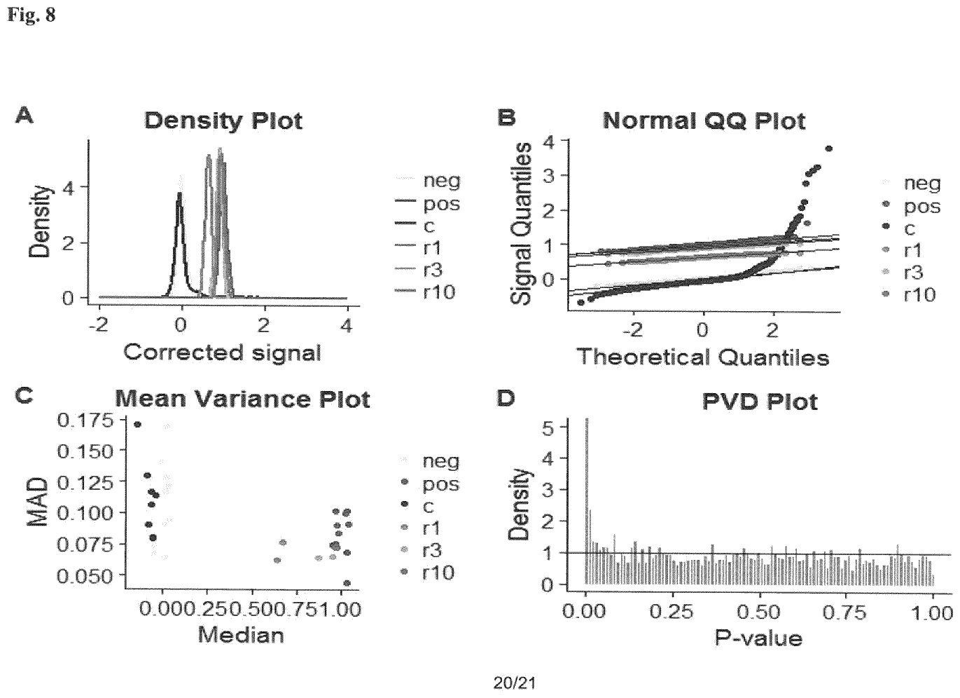

FIG. 8 Distribution of controls and compounds. (A) Empirical probability density, (B) Quantile-Quantile plot, (C) plate-wise MAD vs plate-wise median. (D) P-value distribution (PVD). Regular PVD's can have 2 distinct shapes: (i) uniform if there is no DEG (plateau at density=1). (ii) a peak at small p-values and a plateau <1 for p→1 the plateau value corresponds to the proportion of DEG's. Any other shape is irregular and sensitively indicates a violation of the model assumptions.

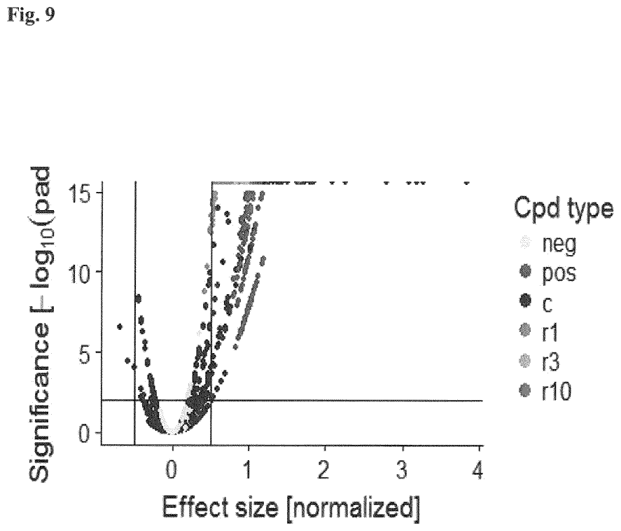

FIG. 9 Volcano plot of all wells display the relation between the significance of the test, expressed as the negative logarithm of the false discovery rate (FDR), and the effect size, expressed as the logarithm of the fold-change. The horizontal line corresponds to FDR=0.01 and cpd's below this line are labeled ‘not significant’. The two vertical lines correspond to a change of 0.5 in activity and cpd's outside this range are labeled ‘strong’.

### EXAMPLE 1

### Method for Determining Development of Resistance

1. Preparation and Ex Vivo Treatment of Tumor Slices
Surplus tumor tissue was stored in cold serum-free RPMI 1620 Glutamax at 4° C. for up to 24 h before processing. Large biopsies were trimmed to about 1 cm maximal diameter.
A 50 ml Falcon tube was cut at the 40 ml mark and the bottom part was discarded. The biopsy was placed into the lid of the shortened Falcon tube. The tube was filled up to the 45 ml mark with with liquid 4% low temperature agarose (SeaPlaque Agarose, Lonza). The agarose was left to solidify on ice.
Once solidified, the agarose block was removed from the Falcon tube and trimmed to a rectangle. About 2 mm of agarose was left from the left and right of the tissue biopsy. About 5 mm of agarose was left from the top and bottom of the tissue biopsy to prevent the tissue from being pushed out of the agarose during the cutting procedure.
The trimmed agarose block was glued onto the specimen holder of a vibratome (Leica VT1000 S Vibrating blade microtome) using cyanoacrylate adhesive and let dry at room temperature. Once dried, the specimen holder was snapped into the buffer tray and the buffer tray installed in the ice bath tray. The buffer tray was filled with ice cold PBS. The biopsy was cut into 400 μm thick slices at a speed of around 0.30 mm/s and a vibration amplitude of about 0.70 mm.
1 nil PRMP 1620 Glutamax containing 1× Antibiotic-Antimycotic (Gibco) and 10% FCS was added to each well of a 6 well plate. One Millicell insert (Millicell Cell Culture Insert, 30 mm, hydrophilic PTFE, 0.4 μm) was transferred into each well. Several slices were transferred onto one Millicell insert. Slices were kept in a humidified cell culture incubator (21% O₂, 5% CO₂) and left to recover for 24 h.
After recovery, Millicell inserts were rinsed by transferring them into a well on a 6 well plate containing 1 ml PRMP 1620 Glutamax (1× Antibiotic-Antimycotic (Gibco), 10% FCS) and either 0.01% DMSO (for control biopsies) or 0.5 μM Selumetinib (for treatment biopsies).

The inserts were then transferred into a fresh well containing 1 ml PRMP 1620 Glutamax (1× Antibiotic-Antimycotic (Gibco), 10% FCS) and either 0.01% DMSO (for control biopsies) or 0.5 μM Selumetinib (for treatment biopsies). Biopsies were incubated for 16 h to 48 h in a humidified cell culture incubator.

2. Gene Expression Analysis of Tumor Slices
To analyze gene expression, tumor slices were transferred into 2.0 ml Eppendorf Safe-Lock microcentrifuge tube (round bottom) using sterile tweezers, weighed, flash frozen in liquid nitrogen and stored at −80° C. until further use. RNA was isolated according to manufacturer's instructions using a RNeasy Mini or Micro Kit (Qiagen) depending on tissue weight and was eluted in 10-30 μl RNase-free water and stored at −80° C. RNA concentration and purity was determined spectrophotometrically using a NanoDrop 2000 (Thermo Scientific). RNA was reverse transcribed to cDNA according to manufacturer's instructions using a High-capacity cDNA Reverse Transcription Kit (Thermo Fisher Scientific). Per 20 μl reaction the following mix was prepared in a Roche LightCycler 480 Multiwell Plate: 3 μl water, 1 μl 5 μM forward primer, 1 μl 5 μM reverse primer, 10 μl LightCycler 480 SYBR Green I Master (2×) or KAPA SYBR® FAST qPCR Kit, 5 μl cDNA [5 ng/μl]. Samples were pipetted in duplicates. 96-well-plates were centrifuged for 1 min at 2000 rpm and run on a LightCylcer 480 device (Roche) using the standard qRT-PCR protocol with 45 cycles. Relative gene expression levels were calculated using the ΔΔCt method {Livak:2001is}, normalizing to GAPDH, TBP or HPRT.

Patients, whose tumor slices express more of a gene associated with the development of resistance as selected from the group comprising SOX, Nanog, OCT4, FGF4, FBX15, FOXP4, KLF9, CD24, CO271, CD36, ITLN2, TNFSFI2, NOX3, CLEC7A, ACYAPI, UNC5C, UNC5D, MUC16, VAV3, FOXD3, VGLL3, ALPP, C3, F2R, ENPP2, ETV4, NTNGI, NTRK2, ROBO1 and ROBO2 after the short-term ex vivo selumetinib treatment as compared to control-treated slices from the same tumor biopsy are likely to develop resistance against selumetinib-based cancer therapies.

3. Immunohistochemical Analysis of Tumor Slices
To prevent damage to the tissue slices, they were lifted out of Millicell inserts using small strips of nitrocellulose membrane. The strips with the tissue slices were then transferred to 4% paraformaldehye. After 24 h, nitrocellulose membrane-attached tumor slices were either transferred to 70% ethanol or directly embedded in paraffin and sectioned according to standard protocols. Paraffin sections were cooked for 20 minutes at 98° C. in EDTA buffer (pH 9) (Dako S2367) using a pressure cooker. Sections were blocked with Peroxidase Block (Dako S2023) for 10 minutes, incubated for 1 h with anti-SOX2 antibody (sc365823) diluted in Dilution Buffer (Dako S2022) to 2 μg/ml, and incubated with the secondary antibody (Envision Mouse, Dako K4001) for 30 minutes. All steps were performed at room temperature. Nuclear counterstain was performed using haematoxylin solution modified according to Gill II (Merck 1051752500) for 2 seconds. After dehydration, slides were coverslipped using a Tissue-Tek Film Coverslipper (Sakura, 4742).
Patients, whose tumor slices express more SOX2 after the short-term ex vivo selumetinib treatment as compared to control-treated slices from the same tumor biopsy are likely to develop resistance against selumetinib-based cancer therapies.

### EXAMPLE 2

### Chemical Compounds for use in Treating Cancer in Combination with a Resistance Inhibitor

1. Screen and Acquisition
On Day 1, A375 cells were seeded at 1400 cell per well into a 384 well plate in DMEM supplemented with 10% FCS and 2 mM L-Glutamine. Plates were kept in a humidified cell culture incubator (21% O₂, 5% CO₂) and left to recover for 16 h. On Day 2, cells were treated with PLX4720 and AZD6244 with a final concentration of 1 uM and 0.5 uM, respectively. At the same time, the cells were treated with a library of FDA-approved drugs (Table 1) with a final concentration of 5 uM. Plates were kept in a humidified cell culture incubator (21% O₂, 5% CO₂) and left to recover for 24 h. On Day 3, plates were three times washed with PBS and fixed with a final concentration of 2% PFA for 10 min at room temperature.
Plates were washed three times with PBS and incubated with a final concentration of 0.2% Triton-X (in PBS) for 10 min at 4° C. Plates were washed three times with PBS and incubated with a final concentration of 0.2% Triton-X (in PBS) for 10 min at 4° C. Plates were washed three times with PBS and incubated with a final concentration of 1% Glycine (in PBS) for 10 min at 4° C. Plates were washed three times with PBS and incubated with a final concentration of 0.8 μg/ml Sox-2 antibody (SantaCruz, E-4, sc-365823) (in 0.05% Tween 20/PBS) overnight. On Day 4, plates were washed three times with PBS. Plates were incubated with a final concentration of 2 μg/ml secondary antibody (A-11029, goat anti-mouse alexa-488, 2 mg/ml) (diluted with CMF-PBS+0.05% Tween 20) for 1 h at room temperature. Plates were washed three times with PBS and incubated with a final concentration of 6.7 ug/ml propidium iodide and 0.2 mg/ml Rnase A fro 1 h at room temperature. After 1 h plates were read with an acumen cellestia (TTP Labtech). For this, fluorophores were excitied at 488 nm and signals were measured in FL3 (nuclear stainging) and FL2 (secondary antibody). The screen was run in duplicate within one month.

2. Preprocessing
Systematic variation from plate to plate is removed by standard normalization procedures (Malo, Nat Biotech, 2006). Following common practice, assay quality is evaluated on the basis of the Z′ factor. Within-plate row-wise or column-wise stripe patterns as well as edge effects can arise during plate preparation. These patterns are eliminated using the median polish method of Tuckey (Tukey, Reading Massachusetts: Addison-Wesley, 1977) or by subtracting a smooth polynomial using the loess function (Boutros, Genome Biology, 2006).

3. Differential Activity Analysis

Differential activity was analyzed following the workflow sketched in Prummer et al (Prummer, J Biomol Screen, 2012). Briefly, for each single-dose measurement of a compound, a Z-test is performed against the Null hypothesis that its activity is indistinguishable from the negative controls. For this to be valid, the distribution of activities of the negative controls are checked to be normal. The mean and variance of the distribution is estimated robustly for each plate and, appropriate, smoothly averaged over a range of consecutive plates.

The semi-automated workflow is implemented in the R environment for statistical computing (Huber, Nat Methods, 2015).

TABLE 1

| Drug. name | CAS. | raw | norm | cor. loess | z | pval | padj |
|---|---|---|---|---|---|---|---|
| DRONEDARONE | 141625-93-6 | 0.87213341 | 4.00362873 | 3.82314631 | 52.0129975 | 0 | 1.42E−13 |
| HYDROCHLORIDE | | | | | | | |
| THIRAM | 137-26-8 | 0.88424461 | 3.60299395 | 3.26613572 | 44.4350007 | 0 | 1.42E−13 |
| PENFLURIDOL | 26864-56-2 | 0.91785544 | 2.4911578 | 2.24830134 | 30.5876057 | 0 | 1.42E−13 |
| PYRITHIONE ZINC | 13463-41-7 | 0.89796146 | 3.23499818 | 3.17051487 | 26.1297191 | 0 | 1.42E−13 |
| THIMEROSAL | 54-64-8 | 0.86629934 | 6.07392047 | 6.42250877 | 25.2453176 | 0 | 9.09E−13 |
| MITOXANTRONE | 70476-82-3, | 0.93739611 | 1.84475834 | 1.83801051 | 25.0056964 | 0 | 1.42E−13 |
| HYDROCHLORIDE | 65271-80-9 [base] | | | | | | |
| LDK378 | 1032900-25-6 | 0.88567874 | 3.41143875 | 3.06496564 | 23.9157992 | 0 | 1.42E−13 |
| PHENYLMERCURIC ACETATE | 62-38-4 | 0.90015274 | 3.16718725 | 2.78289492 | 23.8811952 | 0 | 1.42E−13 |
| THIRAM | 137-26-8 | 0.87061094 | 5.84893751 | 5.61534231 | 22.3535397 | 0 | 9.09E−13 |
| CETRIMONIUM BROMIDE | 57-09-0, 6899-10- | 0.88411767 | 4.7646491 | 5.01373006 | 19.5078759 | 0 | 9.09E−13 |
| | 1 [cetrimonium] | | | | | | |
| OUABAIN OCTAHYDRATE | 11018-89-6 | 0.93870974 | 1.76872708 | 1.68251849 | 18.6531299 | 0 | 1.42E−13 |
| Idarubicin-HCl | 57852-57-0 | 0.94244724 | 1.67654727 | 1.70928639 | 18.2751446 | 0 | 1.42E−13 |
| AZD9291 | 1421373-65-0 | 0.92509565 | 2.16959587 | 2.0825523 | 16.2500689 | 0 | 1.42E−13 |
| Neratinib (HKI-272) | 698387-09-6 | 0.90436083 | 4.1564606 | 4.18160135 | 16.1057509 | 0 | 9.09E−13 |
| PYRITHIONE ZINC | 13463-41-7 | 0.8268055 | 8.51129482 | 8.61807959 | 15.671586 | 0 | 9.09E−13 |
| BENZYL ISOTHIOCYANATE | 622-78-6 | 0.95384043 | 1.25995646 | 1.30518091 | 14.4698018 | 0 | 1.42E−13 |
| CHLOROTHALONIL | 1897-45-6 | 0.90525187 | 4.07310589 | 3.90774183 | 14.0269464 | 0 | 9.09E−13 |
| YM155 (Sepantronium | 781661-94-7 | 0.93418118 | 1.88335309 | 1.79013617 | 13.968358 | 0 | 1.42E−13 |
| Bromide) | | | | | | | |
| VORINOSTAT | 149647-78-9 | 0.96341695 | 0.98399687 | 0.98678747 | 13.4250091 | 0 | 1.42E−13 |
| DIGOXIN | 20830-75-5 | 0.94333408 | 1.66813301 | 1.62310122 | 13.3767482 | 0 | 1.42E−13 |
| SANGUINARIUM CHLORIDE | 5578-73-4 | 0.94498408 | 1.61115323 | 1.49971785 | 12.3598872 | 0 | 1.42E−13 |
| DIGITOXIN | 71-63-6 | 0.94679382 | 1.54865694 | 1.487977 | 12.2631253 | 0 | 1.42E−13 |
| YM155 (Sepantronium | 781661-94-7 | 0.93261472 | 2.64602537 | 3.10768206 | 11.9694703 | 0 | 9.09E−13 |
| Bromide) | | | | | | | |
| EMETINE DIHYDROCHLORIDE | 316-42-7, 483-18- | 0.94850704 | 1.48949379 | 1.4290969 | 11.7778664 | 0 | 1.42E−13 |
| | 1 [emetine] | | | | | | |
| CHLOROTHALONIL | 1897-45-6 | 0.9594557 | 1.11245645 | 1.09494703 | 11.7068243 | 0 | 1.42E−13 |
| DAUNORUBICIN | 20830-81-3 | 0.95153076 | 1.38507484 | 1.3865946 | 11.4275848 | 0 | 1.42E−13 |
| HYDROCHLORIDE | | | | | | | |
| SANGUINARIUM CHLORIDE | 5578-73-4 | 0.87553252 | 5.70391993 | 6.07702228 | 11.0507888 | 0 | 9.09E−13 |
| DACTINOMYCIN | 50-76-0 | 0.93346583 | 1.99004004 | 1.82634262 | 10.8038781 | 0 | 1.42E−13 |
| Quisinostat (JNJ-26481585) | 875320-31-3 | 0.94796667 | 1.44903679 | 1.355698 | 10.578455 | 0 | 1.42E−13 |
| NICLOSAMIDE | 50-65-7 | 0.97004324 | 0.76480106 | 0.72241042 | 9.82822222 | 0 | 1.42E−13 |
| Romidepsin (FK228, | 128517-07-7 | 0.95059614 | 1.399559 | 1.3825506 | 9.78040257 | 0 | 1.42E−13 |
| Depsipeptide) | | | | | | | |
| Bardoxolone Methyl | 218600-53-4 | 0.95125237 | 1.34551965 | 1.24995848 | 9.75337394 | 0 | 1.42E−13 |
| DOXORUBICIN | 23214-92-8 | 0.94429955 | 1.61589439 | 1.61997959 | 9.58312088 | 0 | 1.42E−13 |
| MITOXANTRONE | 70476-82-3, | 0.94442833 | 1.98261811 | 2.3433515 | 9.32840738 | 0 | 9.09E−13 |
| HYDROCHLORIDE | 65271-80-9 [base] | | | | | | |
| PCI-24781 (Abexinostat) | 783355-60-2 | 0.95293628 | 1.29246755 | 1.16573113 | 9.09615145 | 0 | 1.42E−13 |
| LANATOSIDE C | 17575-22-3 | 0.96302575 | 1.04045418 | 1.05905089 | 9.08816239 | 0 | 1.42E−13 |
| Panobinostat (LBH589) | 404950-80-7 | 0.95177866 | 1.32893883 | 1.16285765 | 9.07372984 | 0 | 1.42E−13 |
| SALINOMYCIN, SODIUM | 55721-31-8; | 0.96713651 | 0.81287521 | 0.80196008 | 8.8908774 | 0 | 1.42E−13 |
| | 53003-10-4 (acid) | | | | | | |
| GENTIAN VIOLET | 548-62-9 | 0.96008627 | 1.08962521 | 1.07693072 | 8.87549767 | 0 | 1.42E−13 |
| BROXALDINE | 3684-48-6 | 0.96707764 | 0.81485474 | 0.79925633 | 8.86090246 | 0 | 1.42E−13 |
| TENIPOSIDE | 29767-20-2 | 0.96668415 | 0.87591891 | 0.64457876 | 8.76934098 | 0 | 1.42E−13 |
| Pracinostat (SB939) | 929016-96-6 | 0.95551467 | 1.21123447 | 1.11037175 | 8.66418453 | 0 | 1.42E−13 |
| AZACITIDINE | 320-67-2 | 0.9650662 | 0.97143413 | 0.99449279 | 8.53416216 | 0 | 1.42E−13 |
| Homoharringtonine | 26833-87-4 | 0.95762504 | 1.16115231 | 1.18095408 | 8.35427385 | 0 | 1.42E−13 |
| ACRISORCIN | 7527-91-5 | 0.9751482 | 0.59593073 | 0.58386821 | 7.94338825 | 2.00E−15 | 1.42E−13 |
| Quisinostat (JNJ-26481585) | 875320-31-3 | 0.95298541 | 1.55702088 | 2.04298279 | 7.86870125 | 3.55E−15 | 9.09E−13 |
| Idarubicin-HCl | 57852-57-0 | 0.95670469 | 1.47952809 | 2.18837332 | 7.85522602 | 4.00E−15 | 9.30E−13 |
| TOLONIUM CHLORIDE | 92-31-9 | 0.97100232 | 0.68288694 | 0.70815092 | 7.85086832 | 4.22E−15 | 2.92E−13 |
| Radotinib | 926037-48-1 | 0.9713066 | 0.71941795 | 0.71863124 | 7.68337595 | 1.55E−14 | 1.05E−12 |
| AMODIAQUINE | 6398-98-7, 69-44-3 | 0.96113307 | 1.05347566 | 0.93077195 | 7.6709338 | 1.71E−14 | 1.12E−12 |
| DIHYDROCHLORIDE | [anhydrous], | | | | | | |
| | 86-42-0 | | | | | | |
| | [amodiaquine] | | | | | | |
| BENZETHONIUM CHLORIDE | 121-54-0 | 0.96351605 | 0.97118357 | 0.92876275 | 7.65437498 | 1.93E−14 | 1.24E−12 |
| Chidamide | 743420-02-2 | 0.96178815 | 1.0199474 | 1.06680668 | 7.54677538 | 4.46E−14 | 2.79E−12 |
| CUDC-101 | 1012054-59-9 | 0.96149009 | 1.02297681 | 0.9584341 | 7.47862134 | 7.51E−14 | 4.57E−12 |
| SELAMECTIN | 165108-07-6 | 0.9768345 | 0.54014824 | 0.53298006 | 7.25106711 | 4.13E−13 | 2.46E−11 |
| Tetrandrine | 518-34-3 | 0.96185234 | 1.01777042 | 1.01782421 | 7.20026488 | 6.01E−13 | 3.50E−11 |
| Ispinesib (SB-715992) | 336113-53-2 | 0.93866419 | 2.32262473 | 1.85171565 | 7.13202155 | 9.89E−13 | 2.11E−10 |
| Belirtostat (PXD101) | 414864-00-9 | 0.96057989 | 1.05165301 | 0.89216711 | 6.96154282 | 3.37E−12 | 1.91E−10 |
| Etravirine (TMC125) | 269055-15-4 | 0.97313113 | 0.65890663 | 0.64597988 | 6.90661074 | 4.96E−12 | 2.76E−10 |
| Ispinesib (SB-715992) | 336113-53-2 | 0.96076213 | 1.04591147 | 0.87247738 | 6.80790465 | 9.90E−12 | 5.39E−10 |
| OXYQUINOLINE HEMISULFATE | 134-31-6 | 0.96646147 | 0.86946854 | 0.81755667 | 6.73787289 | 1.61E−11 | 8.57E−10 |
| AMCINONIDE | 51022-69-6 | 0.97609298 | 0.56467769 | 0.49099385 | 6.67985466 | 2.39E−11 | 1.25E−09 |
| PHENYLMERCURIC ACETATE | 62-38-4 | 0.94198189 | 2.09237768 | 1.67992944 | 6.60339343 | 4.02E−11 | 7.91E−09 |
| Panobinostat (LBH589) | 404950-80-7 | 0.9545552 | 1.47310102 | 1.70785251 | 6.57792188 | 4.77E−11 | 8.72E−09 |
| OXIBENDAZOLE | 20559-55-1 | 0.97630095 | 0.55779809 | 0.47811706 | 6.50466898 | 7.79E−11 | 3.99E−09 |
| Belinostat (PXD101) | 414864-00-9 | 0.94883289 | 1.77901194 | 1.67176503 | 6.43892825 | 1.20E−10 | 2.05E−08 |
| ACETYL-L-LEUCINE | 99-15-0 | 0.97369871 | 0.5922208 | 0.57820032 | 6.41017957 | 1.45E−10 | 7.30E−09 |
| CHLOROXINE | 773-76-2 | 0.97575104 | 0.57598873 | 0.47095325 | 6.40720704 | 1.48E−10 | 7.30E−09 |
| Napabucasin | 83280-65-3 | 0.96580916 | 0.88690312 | 0.82040308 | 6.40157103 | 1.54E−10 | 7.43E−09 |
| BENZYL ISOTHIOCYANATE | 622-78-6 | 0.95465515 | 1.41276801 | 1.61684724 | 6.29097595 | 3.15E−10 | 5.05E−08 |
| Mocetinostat (MGCD0103) | 726169-73-9 | 0.95941063 | 1.2135326 | 1.6273413 | 6.26782693 | 3.66E−10 | 5.51E−08 |
| Resminostat | 864814-88-0 | 0.96558788 | 0.89387469 | 0.79996504 | 6.24209393 | 4.32E−10 | 2.05E−08 |
| IDOXURIDINE | 54-42-2 | 0.97259781 | 0.71667087 | 0.71503283 | 6.13599832 | 8.46E−10 | 3.94E−08 |
| TIOGUANINE | 154-42-7 | 0.97544262 | 0.53358164 | 0.54846877 | 6.0805627 | 1.20E−09 | 5.38E−08 |
| THIMEROSAL | 54-64-8 | 0.9726295 | 0.7155989 | 0.70122562 | 6.01751282 | 1.77E−09 | 7.69E−08 |
| CYCLOHEXIMIDE | 66-81-9 | 0.97381236 | 0.67558781 | 0.6971238 | 5.98231342 | 2.20E−09 | 9.39E−08 |
| TRIFLURIDINE | 70-00-8 | 0.97960546 | 0.44848579 | 0.43872651 | 5.96876994 | 2.39E−09 | 1.00E−07 |
| BETAMETHASONE 17,21- | 5593-20-4 | 0.98005692 | 0.43355154 | 0.43562319 | 5.92654998 | 3.09E−09 | 1.28E−07 |
| DIPROPIONATE | | | | | | | |
| Dovitinib (TKI-258) Dilactic | 852433-84-2 | 0.96743384 | 0.83571697 | 0.75656854 | 5.90347283 | 3.56E−09 | 1.45E−07 |
| Acid | | | | | | | |
| COLCHICINE | 64-86-8 | 0.96925119 | 0.77313059 | 0.71106849 | 5.86025318 | 4.62E−09 | 1.85E−07 |
| CUDC-101 | 1012054-59-9 | 0.95976785 | 1.19443618 | 1.51965288 | 5.85305689 | 4.83E−09 | 6.86E−07 |
| Mocetinostat (MGCD0103) | 726169-73-9 | 0.96670941 | 0.85854042 | 0.7471306 | 5.82982895 | 5.55E−09 | 2.19E−07 |
| Sunitinib | 557795-19-4 | 0.97749959 | 0.51402562 | 0.53851743 | 5.75765651 | 8.53E−09 | 3.26E−07 |
| Pelitinib (EKB-569) | 257933-82-7 | 0.96767793 | 0.82802704 | 0.73756467 | 5.75518637 | 8.65E−09 | 3.26E−07 |
| Pimavanserin | 706782-28-7 | 0.968867 | 0.79056507 | 0.73755355 | 5.7550996 | 8.66E−09 | 3.26E−07 |
| EFLOXATE | 119-41-5 | 0.95499681 | 1.39653254 | 1.46838846 | 5.71333908 | 1.11E−08 | 1.49E−06 |
| TG101348 (SAR302503) | 936091-26-8 | 0.96075706 | 1.14155362 | 1.47071537 | 5.66457042 | 1.47E−08 | 1.89E−06 |
| DOXORUBICIN | 23214-92-8 | 0.94397498 | 1.93369629 | 2.35830981 | 5.55413388 | 2.79E−08 | 3.40E−06 |
| Dovitinib (TKI-258) Dilactic | 852433-84-2 | 0.95253573 | 1.58106043 | 1.43565146 | 5.52951914 | 3.21E−08 | 3.74E−06 |
| Acid | | | | | | | |
| PYRVINIUM PAMOATE | 3546-41-6 | 0.94851143 | 1.74886846 | 1.39733131 | 5.49256902 | 3.96E−08 | 4.41E−06 |
| CLOBETASOL PROPIONATE | 25122-46-7, | 0.9793959 | 0.45541808 | 0.40212637 | 5.47083379 | 4.48E−08 | 1.62E−06 |
| | 25122-41-2 | | | | | | |
| | [clobetasol] | | | | | | |
| PCI-24781 (Abexinostat) | 783355-60-2 | 0.96167538 | 1.09246058 | 1.419458 | 5.46714883 | 4.57E−08 | 4.88E−06 |
| METHYLPREDNISOLONE | 03.03.2375 | 0.98056851 | 0.41662847 | 0.40075058 | 5.45211643 | 4.98E−08 | 1.77E−06 |
| SODIUM SUCCINATE | | | | | | | |
| DICHLORISONE ACETATE | 79-61-8 | 0.97714156 | 0.47645455 | 0.49113263 | 5.44490937 | 5.18E−08 | 1.82E−06 |
| ALBENDAZOLE | 54965-21-8 | 0.98109493 | 0.39921444 | 0.39774402 | 5.4112129 | 6.26E−08 | 2.15E−06 |
| Entinostat (MS-275) | 209783-80-2 | 0.96721246 | 0.8426916 | 0.69333717 | 5.41008104 | 6.30E−08 | 2.15E−06 |
| AMINACRINE | 90-45-9, 134-50-9 | 0.96000845 | 1.14402795 | 1.37355309 | 5.39910263 | 6.70E−08 | 6.86E−06 |
| | [aminacrine | | | | | | |
| | hydrochloride] | | | | | | |
| FLUNISOLIDE | 77326-96-6, | 0.97366038 | 0.68072854 | 0.62791371 | 5.38839239 | 7.11E−08 | 2.36E−06 |
| | 3385-03-3 | | | | | | |
| OUABAIN OCTAHYDRATE | 11018-89-6 | 0.95238129 | 1.52081969 | 1.38475854 | 5.38794417 | 7.13E−08 | 7.02E−06 |
| ARTENIMOL | 81496 81-3 | 0.97796256 | 0.44884856 | 0.48292025 | 5.35386342 | 8.61E−08 | 2.83E−06 |
| Ponatinib (AP24534) | 943319-70-8 | 0.97009615 | 0.75184008 | 0.68405408 | 5.33764549 | 9.42E−08 | 3.05E−06 |
| AMINACRINE | 90-45-9, 134-50-9 | 0.97370562 | 0.67919838 | 0.61358023 | 5.26539082 | 1.40E−07 | 4.42E−06 |
| | [aminacrine | | | | | | |
| | hydrochloride] | | | | | | |
| FLUMETHASONE | 2135-17-3 | 0.97358117 | 0.68340787 | 0.60410056 | 5.18404174 | 2.17E−07 | 6.78E−06 |
| AZD9291 | 1421373-65-0 | 0.95730438 | 1.32613148 | 1.34471365 | 5.17926536 | 2.23E−07 | 2.11E−05 |
| Rocihnostat (ACY-1215) | 1316214-52-4 | 0.97054266 | 0.73777259 | 0.65765591 | 5.13166167 | 2.87E−07 | 8.86E−06 |
| BRONOPOL | 52-51-7 | 0.97791383 | 0.450487 | 0.45067331 | 4.99635985 | 5.84E−07 | 1.78E−05 |
| GRAMICIDIN (gramicidin A | 1405-97-6 | 0.97053154 | 0.72891573 | 0.59921976 | 4.93845468 | 7.87E−07 | 2.32E−05 |
| shown) | | | | | | | |
| ABAMECTIN (avermectin B1a | 71751-41-2 | 0.97789413 | 0.5009403 | 0.46062791 | 4.92488663 | 8.44E−07 | 2.46E−05 |
| shown) | | | | | | | |
| ACRISORCIN | 7527-91-5 | 0.96747792 | 0.77535429 | 1.22380691 | 4.87172727 | 1.11E−06 | 9.77E−05 |
| DISULFIRAM | 97-77-8 | 0.97304762 | 0.6420274 | 0.59093753 | 4.8701969 | 1.11E−06 | 3.21E−05 |
| Difluprednate | 23674-86-4 | 0.98005569 | 0.4292515 | 0.45288421 | 4.84209348 | 1.28E−06 | 3.65E−05 |
| ACETRIAZOIC ACID | 85-36-9, 129-63-5 | 0.98168618 | 0.37965601 | 0.35366324 | 4.81150439 | 1.50E−06 | 4.21E−05 |
| | [acetrizoate | | | | | | |
| | sodium] | | | | | | |
| ISOFLUPREDONE ACETATE | 338-98-7 | 0.97904025 | 0.46718275 | 0.35254412 | 4.79627899 | 1.62E−06 | 4.45E−05 |
| LY2835219 | 1231930-82-7 | 0.96296373 | 1.02358655 | 1.2344393 | 4.75453542 | 1.99E−06 | 0.00016973 |
| PERHEXILINE MALEATE | 6724 53-4, 6621- | 0.97862607 | 0.48088372 | 0.3464258 | 4.7130407 | 2.44E−06 | 6.65E−05 |
| | 47-2 [perhexiline] | | | | | | |
| METERGOUNE | 17692-51-2 | 0.97934661 | 0.40230977 | 0.42089336 | 4.66620643 | 3.07E−06 | 8.27E−05 |
| PACLITAXEL | 33069-62-4 | 0.98257921 | 0.35011491 | 0.34103977 | 4.63976513 | 3.49E−06 | 9.30E−05 |
| FORMESTANE | 566-48-3 | 0.9799552 | 0.38184606 | 0.41788935 | 4.63290263 | 3.61E−06 | 9.52E−05 |
| MONENSIN SODIUM | 22373-78-0 | 0.97614292 | 0.59675448 | 0.53594708 | 4.59918801 | 4.24E−06 | 0.0001108 |
| Pracinostat (SB939) | 929016-96-6 | 0.9670443 | 0.80544144 | 1.18174429 | 4.55157667 | 5.32E−06 | 0.00042596 |
| FLOXURIDINE | 50-91-9 | 0.97952104 | 0.45127834 | 0.33437653 | 4.54911331 | 5.39E−06 | 0.00013931 |
| PREDNICARBATE | 73771-04-7 | 0.978218 | 0.4402593 | 0.39916716 | 4.42534034 | 9.63E−06 | 0.00024167 |
| DEXAMETHASONE SODIUM | 2392-39-4, 312- | 0.97479316 | 0.58174824 | 0.52990085 | 4.36716464 | 1.26E−05 | 0.00031284 |
| PHOSPHATE | 93-6 | | | | | | |
| | [dexamethasone | | | | | | |
| | 21-(dihydrogen | | | | | | |
| | phosphate)] | | | | | | |
| LEFLUNOMIDE | 75706-12-6 | 0.98158264 | 0.38308121 | 0.31943653 | 4.34585809 | 1.39E−05 | 0.00034149 |
| Halobetasol Propionate | 66852-54-8 | 0.98094293 | 0.39982608 | 0.40548008 | 4.33526364 | 1.46E−05 | 0.00035495 |
| SIROLIMUS | 53123-88-9 | 0.98084386 | 0.40751985 | 0.31710492 | 4.31413716 | 1.60E−05 | 0.00038335 |
| IPRIFLAVONE | 35212-22-7 | 0.98071348 | 0.35634856 | 0.38850276 | 4.30711018 | 1.65E−05 | 0.00039206 |
| Nintedanib (BIBF 1120) | 656247-17-5 | 0.97437333 | 0.61708628 | 0.54849154 | 4.27985661 | 1.87E−05 | 0.00043922 |
| PYRVINIUM PAMOATE | 3546-41-6 | 0.9688179 | 0.84452979 | 0.49804436 | 4.27392872 | 1.92E−05 | 0.00044697 |
| RUFLOXACIN HYDROCHLORIDE | 106017-08-7, | 0.97961558 | 0.44384801 | 0.399135 | 4.2674241 | 1.98E−05 | 0.00045606 |
| | 101363-10-4 | | | | | | |
| Fosbretabulin (Combretastatin | 168555-66-6 | 0.98140874 | 0.38437733 | 0.39809792 | 4.25633595 | 2.08E−05 | 0.0004726 |
| A4 Phosphate (CA4P)) | | | | | | | |
| Disodium | | | | | | | |
| TRIAMCINOLONE DIACETATE | 67-78-7 | 0.979412 | 0.45059983 | 0.39636101 | 4.23776541 | 2.26E−05 | 0.00050696 |
| ISOFLUPREDONE ACETATE | 338-98-7 | 0.96252984 | 1.0345188 | 1.06197867 | 4.22752184 | 2.36E−05 | 0.0018213 |
| LANATOSIDE C | 17575-22-3 | 0.97234744 | 0.49489242 | 1.0741534 | 4.22223536 | 2.42E−05 | 0.0018213 |
| BROXALDINE | 3684-48-6 | 0.96238556 | 1.0454252 | 1.06866967 | 4.15807692 | 3.21E−05 | 0.00234743 |
| Otenabant (CP-945598) HCl | 686347-12-6 | 0.96647863 | 0.83568218 | 1.06864551 | 4.11596824 | 3.86E−05 | 0.0026812 |
| DAUNORUBICIN | 20830-81-3 | 0.9308753 | 2.51538168 | 2.26279979 | 4.11479857 | 3.88E−05 | 0.0026812 |
| HYDROCHLORIDE | | | | | | | |
| Aprotinin | 9087-70-1 | 0.96622985 | 0.99939391 | 1.14018378 | 4.09272092 | 4.26E−05 | 0.00282021 |
| Romidepsin (FK228, | 128517-07-7 | 0.96814302 | 0.86910378 | 1.80622573 | 4.09093323 | 4.30E−05 | 0.00282021 |
| Depsipeptide) | | | | | | | |
| FLUTICASONE PROPIONATE | 80474-14-2 | 0.97044386 | 0.71299307 | 0.68747124 | 4.06679196 | 4.77E−05 | 0.00106106 |
| CHLOROXINE | 773-76-2 | 0.96835735 | 0.7292924 | 1.01834333 | 4.05381842 | 5.04E−05 | 0.00322485 |
| Amuvatinib (MP-470) | 850879-09-3 | 0.97481609 | 0.60313686 | 0.51909338 | 4.05046399 | 5.11E−05 | 0.0011258 |
| METHYLBENZETHONIUM | 1320-44-1, | 0.98263238 | 0.34835613 | 0.29761114 | 4.04892884 | 5.15E−05 | 0.0011258 |
| CHLORIDE | 25155-18-4 | | | | | | |
| | [anhydrous] | | | | | | |
| FENBENDAZOLE | 43210-67-9 | 0.98084656 | 0.43765002 | 0.47094603 | 4.04138654 | 5.31E−05 | 0.00115278 |
| BETAMETHASONE | 378-44-9 | 0.97187427 | 0.51978516 | 1.01640799 | 3.99525223 | 6.46E−05 | 0.00403515 |
| BUPIVACAINE | 14252-80-3 | 0.98350106 | 0.31962038 | 0.2933597 | 3.99108906 | 6.58E−05 | 0.0014149 |
| HYDROCHLORIDE | | | | | | | |
| DACTINOMYCIN | 50-76-0 | 0.93875623 | 2.20141609 | 1.68790698 | 3.97524588 | 7.03E−05 | 0.00428535 |
| BETAMETHASONE | 378-44-9 | 0.98099496 | 0.43263023 | 0.46233628 | 3.96750266 | 7.26E−05 | 0.00153855 |
| FLUMETHAZONE PIVALATE | 2002-29-1, | 0.97664178 | 0.51790904 | 0.4808963 | 3.96329484 | 7.39E−05 | 0.00153855 |
| | 2135-17-3 | | | | | | |
| | [flumethasone] | | | | | | |
| THIOGUANINE | 154-42-7, | 0.96733045 | 0.75882881 | 0.99887156 | 3.9263208 | 8.63E−05 | 0.00513519 |
| | 5580-03-0 | | | | | | |
| | [hemihydrate] | | | | | | |
| TEGASEROD MALEATE | 189188-57-6 | 0.98485704 | 0.27476498 | 0.28641845 | 3.89665499 | 9.75E−05 | 0.00199742 |
| PREDNISOLONE ACETATE | 52-21-1 | 0.9771695 | 0.49968527 | 0.46966514 | 3.87073354 | 0.00010851 | 0.00220461 |
| CHLORINDIONE | 1146-99-2 | 0.98249161 | 0.29655912 | 0.34626428 | 3.83883607 | 0.00012362 | 0.00249185 |
| HYDROCORTISONE | 83784-20-7, | 0.9769567 | 0.50703385 | 0.46222171 | 3.80938871 | 0.00013931 | 0.00278622 |
| HEMISUCCINATE | 2203-97-6 | | | | | | |
| | [anhydrous] | | | | | | |
| DEXAMETHASONE ACETATE | 55812-90-3, | 0.97690425 | 0.50884539 | 0.46148467 | 3.8033144 | 0.00014277 | 0.00281153 |
| | 1177-87-3 | | | | | | |
| | [anhydrous] | | | | | | |
| FLUDROCORTISONE ACETATE | 514-36-3, | 0.97697735 | 0.50632098 | 0.45776169 | 3.77263151 | 0.00016153 | 0.0031328 |
| | 127-31-1 | | | | | | |
| | [fludrocortisone] | | | | | | |
| IVERMECTIN | 70288-86-7 | 0.97257709 | 0.63932157 | 0.63580123 | 3.76113383 | 0.00016914 | 0.00325572 |
| PROFLAVINE HEMISULFATE | 553-30-0, | 0.98279968 | 0.28619997 | 0.3381165 | 3.74850618 | 0.00017789 | 0.00339851 |
| | 1811-28-5 | | | | | | |
| LANSOPRAZOLE | 103577-45-3 | 0.98194159 | 0.37120734 | 0.27321749 | 3.71705911 | 0.00020156 | 0.00382209 |
| Cerdulatinib (PRT062070, | 1369761-01-2 | 0.97412848 | 0.6248001 | 0.4671962 | 3.64551246 | 0.00026686 | 0.00495044 |
| PRT2070) | | | | | | | |
| HALCINONIDE | 3093-35-4 | 0.98534639 | 0.25857767 | 0.2631958 | 3.58071637 | 0.00034265 | 0.00622123 |
| TERFENADINE | 50679-08-8 | 0.97666195 | 0.54585633 | 0.26014742 | 3.53924389 | 0.00040127 | 0.00716911 |
| FLUOCINONIDE | 356-12-7 | 0.9796993 | 0.41232308 | 0.42928475 | 3.53793951 | 0.00040326 | 0.00716911 |
| HEXETIDINE | 141-94-6 | 0.98342817 | 0.26506696 | 0.31545283 | 3.49724691 | 0.00047009 | 0.00824261 |
| ARTESUNATE | 182824-33-5 | 0.98353492 | 0.31386196 | 0.32464253 | 3.47097433 | 0.00051857 | 0.00896992 |
| FLUOCINOLONE ACETONIDE | 67-73-2 | 0.97907225 | 0.43397706 | 0.4201774 | 3.46288151 | 0.00053442 | 0.00912083 |
| BENZOXIQUINE | 86-75-9 | 0.97314583 | 0.61967992 | 0.58457551 | 3.45810394 | 0.00054399 | 0.00922264 |
| RIFAMPIN | 13292-46-1 | 0.97922438 | 0.49252149 | 0.40252899 | 3.45427103 | 0.00055178 | 0.00929318 |
| TRIAMCINOLONE | 124-94-7 | 0.98213259 | 0.39414892 | 0.400624 | 3.43792354 | 0.00058619 | 0.0098082 |
| ZOLPIDEM | 82626-48-0 | 0.97982598 | 0.44119093 | 0.2522452 | 3.43173598 | 0.00059973 | 0.00996956 |
| NOCODAZOLE | 31430-18-9 | 0.97658546 | 0.50089146 | 0.53110307 | 3.14178334 | 0.00167922 | 0.02356102 |
| TRICLOSAN | 3380-34-5 | 0.97534408 | 0.54376266 | 0.53095524 | 3.14090884 | 0.00168424 | 0.02356102 |
| METHYLENE BLUE | 7220-79-3 | 0.97507511 | 0.55305178 | 0.51931963 | 3.07207749 | 0.00212575 | 0.02894632 |
| PROSCILLARIDIN A | 466-06-8 | 0.9786549 | 0.42942253 | 0.50832339 | 3.0070283 | 0.00263815 | 0.03410944 |

1. Method for the determination whether a cancer or tumor cell will develop resistance to a chemical substance, wherein the method comprises the following steps:
a) exposure of one or more sample(s) comprising or consisting of cancer or tumor cells obtained from a subject diagnosed with cancer to a chemical substance, wherein the subject diagnosed with cancer has not previously been administered with the chemical substance;
b) determining the expression level of a gene associated with the development of cancer drug resistance in the one or more sample(s) used in a);
c) determining the expression level of the same gene as in b) in the one or more sample(s) from the subject diagnosed with cancer that is (are) not exposed to the chemical substance used in a);
wherein an elevated expression level determined in b) compared to the expression level determined in c) is indicative of the development of resistance to the chemical substance by a cancer or tumor cell comprised in said sample.

2. A method for determining whether a subject previously diagnosed with cancer will develop resistance to a chemical substance used for treating said cancer, wherein the method comprises the following steps:
a) exposure of one or more sample(s) comprising or consisting of cancer of tumor cells obtained from the subject diagnosed with cancer to a chemical substance, wherein the subject diagnosed with cancer has not previously been administered with the chemical substance;
b) determining the expression level of a gene associated with the development of cancer drug resistance in the one or more sample(s) used in a);
c) determining the expression level of the same gene as in b) in the one or more sample(s) from the subject diagnosed with cancer that is (are) not exposed to the chemical substance used in a);
wherein the subject diagnosed with cancer has not been administered with the chemical substance used in a) prior to obtaining the one or more sample(s) and wherein an elevated expression level determined in b) compared to the expression level determined in c) is indicative of the development of resistance to the chemical substance by the patient.

3. The method of claim 1 or claim 2, wherein the one or more samples) is obtained by biopsy.

4. The method of claim 1 or claim 2, wherein the sample is obtained from circulating tumor cells in the blood.

5. The method of any one of the preceding claims, wherein the gene associated with the development of resistance is a gene selected from the group consisting of SOX2, Nanog, OCT4, FGF4, FBX15, FOXP4, KLF9, CD24, CD271, CD36, ITLN2, TNFSF12, NOX3, CLEC7A, ACYAP1, UNC5C, UNC5D, UC16, VAV3, FOXD3, VGLL3, ALPP C3, F2R ENPP2 ETV4, NTNG1 NTRK2, ROBO1 and ROBO2.

6. The method of claim 5 wherein the gene associated with the development of resistance is SOX2.

7. The method of any one of the preceding claims, wherein cancer is non-melanoma skin cancer, esophagogastric adenocarcinoma, glioblastoma, bladder cancer, bladder urothelial carcinoma, esophagogastric cancer, melanoma, non-small cell lung cancer, endometrial cancer, cervical adenocarcinoma, esophageal squamous cell carcinoma, breast cancer, head and neck squamous cell carcinoma, germ cell tumor, small cell lung cancer, ovarian cancer, soft tissue sarcoma, hepatocellular carcinoma, colorectal adenocarcinoma, cervical squamous cell carcinoma, cholangiocarcinoma, prostate cancer, upper tract urothelial carcinoma, diffuse glioma, colorectal cancer, ampullary carcinoma, adrenocortical carcinoma, head and neck cancer, renal clear cell carcinoma, hepatobiliary cancer, glioma, non-Hodgkin lymphoma, mesothelioma, salivary gland cancer, renal non-clear cell carcinoma, miscellaneous neuroepithelial tumor, pheochromocytoma, thymic tumor, multiple myeloma, renal cell carcinoma, bone cancer, pancreatic cancer, leukemia, peripheral nervous system tumors, thyroid cancer, B-lymphoblast leukemia, monoclonal B-cell lymphocytosis, lymphoma, hairy cell leukemia, acute myeloid leukemia, Wilms tumor in particular melanoma and non-small cell lung cancer.

8. The method of any one of the preceding claims, wherein said chemical substance is an inhibitor of a receptor tyrosine kinase (RTK), the EGFR pathway (EGFRi) or an inhibitor of the MAPK pathway (MAPKi), wherein, preferably, said MAPKi is an inhibitor of B-Raf (BRAFi), an inhibitor of MEK (MEKi), or an inhibitor of ERK (ERKi).

9. The method according to claim 8, wherein said
i) said BRAFi is vemurafenib, dabrafenib, encorafenib, LGX818, PLX4720, TAK-632, MLN2480, SB590885, XL281, BMS-908662, PLX3603, RO5185426, GSK2118436 or RAF265,
ii) said MEKi is AZD6244, trametinib, selumetinib, cobimetinib, binimetinib, MEK162, RO5126766, GDC-0623, PD 0325901, CI-1040, PD-035901, hypothemycin or TAK-733,
iii) said ERKi is ulixertinib, corynoxeine, SCH772984, XMD8-92, FR 180204, GDC-0994, ERK5-IN-1, DEL-22379, BIX 02189, ERK inhibitor (CAS No. 1049738-54-6), ERK inhibitor III (CAS No. 331656-92-9), GDC-0994, honokiol, LY3214996, CC-90003, deltonin, VRT752271, TIC10, astragaloside IV, XMD8-92, VX-11e, mogrol, or X11e, and/or
iv) said EGFRi is cetuximab, panitumumab, zalutuumab, nimotuzumab, matuzumab, gefitinib, erlotinib, lapatinib, neratinib, vandetanib, necitumumab, osimertinib, afatinib, AP26113, EGFR inhibitor (CAS No. 879127-07-8), EGFR/ErbB-2/ErbB-4 Inhibitor (CAS No. 881001-19-0), EGFR/ErbB-2 Inhibitor (CAS No. 179248-61-4), EGFR inhibitor II (BIBX 1382,CAS No. 196612-93-8), EGFR inhibitor III (CAS No. 733009-42-2), EGFR/ErbB-2/ErbB-4 Inhibitor II (CAS No. 944341-54-2) or PKCβII/EGFR Inhibitor (CAS No. 145915-60-2).

10. A chemical substance for use in treating cancer in patients determined to develop resistance to said chemical substance using the method of any one of claims 1 to 7, in combination with a second chemical substance, wherein said second chemical substance inhibits expression of a gene associated with the development of cancer drug resistance.

11. Use of one or more chemical substances for treating patients determined to developed resistance to said chemical substances using the methods of any one of claims 1-7 in combination with a further chemical substance which inhibits the expression of one or more genes associated with the development of cancer drug resistance to the first chemical substance or substances.

12. A product containing a combination of one or more chemical substances determined to induce cancer drug resistance in a cancer of tumor cell using the methods of any one of claims 1-7 and a further chemical substance which inhibits the expression of one or more genes associated with the development of cancer drug resistance to the first chemical substance.

13. The chemical substance for use of claim 10, the use of claim 11 and the product of claim 12, wherein cancer is non-melanoma skin cancer, esophagogastric adenocarcinoma, glioblastoma, bladder cancer, bladder urothelial carcinoma, esophagogastric cancer, melanoma, non-small cell lung cancer, endometrial cancer, cervical adenocarcinoma, esophageal squamous cell carcinoma, breast cancer, head and neck squamous cell carcinoma, germ cell tumor, small cell lung cancer, ovarian cancer, soft tissue sarcoma, hepatocellular carcinoma, colorectal adenocarcinoma, cervical squamous cell carcinoma, cholangiocarcinoma, prostate cancer, upper tract urothelial carcinoma, diffuse glioma, colorectal cancer, ampullary carcinoma, adrenocortical carcinoma, head and neck cancer, renal clear cell carcinoma, hepatobiliary cancer, glioma, non-Hodgkin lymphoma, mesothelioma, salivary gland cancer, renal non-clear cell carcinoma, miscellaneous neuroepithelial tumor, pheochromocytoma, thymic tumor, multiple myeloma, renal cell carcinoma, bone cancer, pancreatic cancer, leukemia, peripheral nervous system tumors, thyroid cancer, B-lymphoblast leukemia, monoclonal B-cell lymphocytosis, lymphoma, hairy cell leukemia, acute myeloid leukemia, Wilms tumor in particular melanoma and non-small cell lung cancer.

14. The chemical substance for use of claim 10 or claim 13, the use of claim 11 or claim 13 and the product of claim 12 or claim claim 13, wherein said chemical substance is an inhibitor of a receptor tyrosine kinase (RTK), the EGFR pathway (EGFRi), an inhibitor of the MAPK pathway (MAPKi) or an agent used in immunotherapy of cancer, wherein, preferably, said MAPKi is an inhibitor of B-Raf (BRAFi), an inhibitor of MEK (MEKi), or an inhibitor of ERK (ERKi).

15. The chemical substance for use, the use and the product of claim 14, wherein said
i) said BRAFi is vemurafenib, dabrafenib, encorafenib, LGX818, PLX4720, TAK-632, LN2480, SB590885, XL281, BMS-908662, PLX3603, RO5185426, GSK2118436 or RAF265,
ii) said MEKi is AZD6244, trametinib, selumetinib, cobimetinib, binimetinib, MEK162, RO5126766, GDC-0623, PD 0325901, CI-1040, PD-035901, hypothemycin or TAK-733,
iii) said ERKi is ulixertinib, corynoxeine, SCH772984, XMD8-92, FR 180204, GDC-0994, ERK5-IN-1, DEL-22379, BIX 02189, ERK inhibitor (CAS No. 1049738-54-6), ERK inhibitor III (CAS No, 331656-92-9), GDC-0994, honokiol, LY3214996, CC-90003, deltonin, VRT752271, TIC10, astragaloside IV, XMD8-92, VX-11e, mogrol, or VTX11e,
iv) said EGFRi is cetuximab, panitumumab, zalutumumab, nimotuzumab, matuzumab, gefitinib, erlotinib, lapatinib, neratinib, vandetanib, necitumumab, osimertinib, afatinib, AP26113, EGFR inhibitor (CAS No. 879127-07-8), EGFR/ErbB-2/ErbB-4 Inhibitor (CAS No. 881001-19-0), EGFR/ErbB-2 Inhibitor (CAS No. 179248-61-4), EGFR inhibitor II (BIBX 1382,CAS No. 196612-93-8), EGFR inhibitor III (CAS No. 733009-42-2), EGFR/ErbB-2/ErbB-4 Inhibitor II (CAS No. 944341-54-2) or PKCβII/EGFR Inhibitor (CAS No. 145915-60-2); and/or
v) said agent used in immunotherapy is an agent targeting CD52, PD-L1, CTLA4, CD20, or PD-1. Agents that may be used in combination with a compound of the present invention include, for example, alemtuzumab, atezolizumab, ipilimumab, nivolumab, ofatumumab, pembrolizumab, rituximab.

16. The chemical substance for use of any one of claims 10, and 13 to 15, the use of any one of claims 11 and 13 to 15 and the product any one of claims 12 and 13 to 15, wherein said second chemical substance inhibiting expression of a gene associated with the development of cancer drug resistance inhibits a gene selected from the group consisting of SOX2, Nanog, OCT4, FGF4, FBX15, FOXP4, KLF9, CD24, CD271, CD36, ITLN2, TNFSF12, NOX3, CLEC7A, ACYAP1, UNCSC, UNC5D, MUC16, VAV3, FOXD3, VGLL3, ALPP, C3, F2R, ENPP2, ETV4, NTNG1, NTRK2, ROBO1 and ROBO2.

17. The chemical substance for use, the use and the product of claim 16, wherein the gene associated with the development of resistance is SOX2.

18. The chemical substance for use of any one of claims 10 and 13 to 18, the use of any one of claims 11 and 13 to 18 and the product any one of claims 12 and 13 to 18, wherein said second chemical substance is selected from the group consisting of cetrimonium bromide, idarubicin.hcl, neratinib (hki-272), benzyl isothiocyanate, vorinostat, emetine dihydrochloride, daunorubicin hydrochloride, dactinomycin, quisinostat (jnj26481585), niclosamide, doxorubicin, pci-24781 (abexinostat), lanatoside c, panobinostat (lbh589), salinomycin, sodium, broxaldine, teniposide, pracinostat (sb939), azacitidine, homoharringtonine, acrisorcin, tolonium chloride, radotinib, amodiaquine dihydrochloride, benzethonium chloride, chidamide, cudc-101, selamectin, tetrandrine, belinostat (pxd101), etravirine (tmc125), amcinonide, oxibendazole, acetyl-l-leucine, chloroxine, napabucasin, resminostat, idoxuridine, tioguanine, cycloheximide, trifiuridine, betamethasone 17,21, dipropionate, dovitinib (tki-258) dilactic acid, colchicine, mocetinostat (mgcd0103), sunitinib, pelitinib (ekb-569), pimavanserin , efloxate, tg101348 (sar302503), clobetasol propionate, methylprednisolone sodium succinate, dichlorisone acetate, albendazole, entinostat (ms-275), flunisolide, artenimol, aminacrine, flumethasone, rocilinostat (acy-1215), bronopol, gramicidin (gramicidin a shown), abamectin (avermectin b1a shown), disulfiram, difluprednate, acetriazoic acid, isoflupredone acetate, ly2835219, perhexiline maleate, metergoline, formestane, monensin sodium, floxuridine, prednicarbate, dexamethasone sodium phosphate, leflunomide, halobetasol propionate, sirolimus, ipriflavone, nintedanib (bibf 1120), pyrvinium, pamoate, rufloxacin hydrochloride, fosbretabulin (combretastatin a4 phosphate (ca4p)), disodium, triamcinolone diacetate, otenabant (cp-945598) hcl, aprotinin, fluticasone propionate, amuvatinib (mp-470), methylbenzethonium chloride, fenbendazole, bupivacaine hydrochloride, betamethasone, flumethazone pivalate, thioguanine, tegaserod maleate, prednisolone acetate, chlorindione, hydrocortisone hemisuccinate, dexamethasone acetate, fludrocortisone acetate, ivermectin, proflavine hemisulfate, lansoprazole, cerdulatinib (prt062070, prt2070), salifungin, halcinonide, fudosteine, terfenadine, fluocinonide, hexetidine, artesunate, fluocinolone acetonide, rifampin, triamcinolone, zolpidem, ethopropazine, hydrochloride, regorafenib (bay 73-4506), terazosin hydrochloride, tanshinone iia-sulfonic sodium, nocodazole, triclosan, clopidol, sorafenib tosylate, sulfisomidine, methylene blue, crizotinib (pf-02341066), proscillaridin a, dexibuprofen, triflupromazine hydrochloride, piribedil hydrochloride, carmofur, swertiamarin, sultamicillin tosylate, ginsenoside rc, etofibrate, cetylpyridinium chloride, rabeprazole sodium, alizapride hydrochloride, methyl aminolevulinate.hcl, topiroxostat, disodium clodronate tetrahydrate, amoxapine, bedaquiline(tmc207; r207910), octenidine, ecabet sodium, apigenin, glycopyrrolate iodide, sodium montmorillonite, hydrocortisone, barbadin, CCS1477, SGC-CBP30, CPI-637, PF-CBP1, ICG,001,PRI-724, A-485, C646, 4-methylthio-2-oxobutyric acid (MTOB), HIPP derivatives, cyclic peptide CP61, NSC95397, 2-(hydroxyimino)-3-phenylpropanoic acid and 4-chloro and 3-chloro analogues thereof, MLN4924, AS1842856, JIB-04, EP-5676, N-oxalylglycine (NOG), pyridine-2,4-dicarboxylate (2,4-PDCA), pladineolide B, IDC16 CBL0137, difopein, R18.
